SEQUENCE LISTING (20 sequences)

SEQ ID NO: 1 - protein, 5 aa, synthetic construct
NYWIG

SEQ ID NO: 2 - protein, 17 aa, synthetic construct
IIDPSNSYTRYSPSFQG

SEQ ID NO: 3 - protein, 8 aa, synthetic construct
WYYKPFDV

SEQ ID NO: 4 - protein, 14 aa, synthetic construct
TGSSSNIGSGYDVH

SEQ ID NO: 5 - protein, 7 aa, synthetic construct
GNSKRPS

SEQ ID NO: 6 - protein, 11 aa, synthetic construct
ASWTDGLSLVV

SEQ ID NO: 7 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 8 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 9 - protein, 447 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 5 aa, synthetic construct
SYAMH

SEQ ID NO: 12 - protein, 17 aa, synthetic construct
FMSYDGSNKKYADSVKG

SEQ ID NO: 13 - protein, 17 aa, synthetic construct
DRGIAAGGNYYYYGMDV

SEQ ID NO: 14 - protein, 11 aa, synthetic construct
RASQSVYSYLA

SEQ ID NO: 15 - protein, 7 aa, synthetic construct
DASNRAT

SEQ ID NO: 16 - protein, 10 aa, synthetic construct
QQRSNWPPFT

SEQ ID NO: 17 - protein, 126 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 18 - protein, 111 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTV

SEQ ID NO: 19 - protein, 456 aa, synthetic construct
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 215 aa, synthetic construct
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0308243 A1
(43) Pub. Date: Oct. 8, 2026

(54) METHOD OF TREATING CROHN'S DISEASE WITH A COMBINATION OF ANTIBODIES TO IL-23 AND TNF ALPHA

(71) Applicant: Janssen Biotech, Inc., Horsham, PA (US)

(72) Inventors: Marion Vetter, Spring House, PA (US); Natalie A. Terry, Spring House, PA (US); Terence Rooney, Spring House, PA (US); Meredith Hans Moore, Spring House, PA (US); Yan Xu, Spring House, PA (US); Barry Todd Yeager, Spring House, PA (US)

(21) Appl. No.: 19/480,545

(22) PCT Filed: May 2, 2024
(86) PCT No.: PCT/IB2024/054245

§ 371 Date: Oct. 31, 2025

Related U.S. Application Data
(60) Provisional application No. 63/463,658, filed on May 3, 2023.

(51) Int. Cl. C07K 16/24; A61K 9/00; A61K 39/00; A61P 37/06

(52) U.S. Cl. CPC C07K 16/241; A61K 9/0019; A61P 37/06; C07K 16/244; A61K 2039/507; A61K 2039/54; A61K 2039/545; C07K 2317/565

(57) ABSTRACT

A method of treating Crohn's disease (CD) by administering a combination of an IL-23 inhibitor, such as an anti-IL-23p19 antibody (e.g., guselkumab), and a TNF-α inhibitor, such as an anti-TNF-α antibody (e.g., golimumab).

### CROSS-REFERENCE TO RELATED APPLICATIONS

This application claims priority to U.S. Provisional Application Ser. No. 63/463,658, filed May 3, 2023, the entire contents of which are incorporated herein by reference in their entirety.

### REFERENCE TO SEQUENCE LISTING SUBMITTED ELECTRONICALLY

This application contains a sequence listing, which is submitted electronically in XML format and is hereby incorporated by reference in its entirety. Said XML copy, created on Mar. 8, 2024, is named “JBI6806WOPCT1_SL.xml” and is 19,936 bytes in size.

### FIELD OF INVENTION

The present invention concerns methods and kits for treating Crohn's disease with a combination of an IL-23 inhibitor and a TNF-α inhibitor. In particular, it relates to a method of administering an anti-IL-23p19 antibody, e.g., guselkumab, and an anti-TNF-α antibody, e.g., golimumab, to patients suffering from Crohn's disease.

### BACKGROUND

Crohn's disease (CD) is a chronic, progressive, and potentially life-threatening disorder that may affect any part of the gastrointestinal (GI) tract. Symptoms may include diarrhea, abdominal pain (AP), weight loss/anorexia, and fatigue. Inflammation can cause mucosal ulcerations to form and can also penetrate the lining of the GI tract, causing intra-abdominal fistulas, in addition to causing painful perianal disease. CD may also be stricturing, such that patients may require repeated surgeries.

The pathophysiology of inflammatory bowel disease (IBD), including CD, is complex and thought to be multifactorial. The primary aim of pharmacotherapy is to dampen the inflammatory response, thereby relieving symptoms and promoting mucosal healing. The specific goals of IBD treatment include control of symptoms, prevention of relapses and complications, and surveillance of malignant transformation (D'Haens G R et al., Future directions in inflammatory bowel disease management. J Crohns Colitis. 2014; 8 (8): 726-734).

Over the past 20 years, biologic therapies, such as anti-TNF-α, IL-12/23 antagonists, and anti-integrins, have revolutionized the clinical management of IBD. Most agents in these classes are approved for the treatment of both Crohn's disease (CD) and ulcerative colitis (UC). Within the anti-TNF-α class, infliximab and adalimumab are approved for both indications. Golimumab, which is an anti-TNF-α therapy approved in UC, has not previously been evaluated in CD. However, the anti-TNF-α mechanism is well-established for the treatment of CD (D'Haens G R et al., 25 years of anti-TNF treatment for inflammatory bowel disease: lessons from the past and a look to the future [published online ahead of print, 2021 Jan. 11]. Gut. 2021; gutjnl-2019-320022). Ustekinumab, an IL-12/23 antagonist, and vedolizumab, an anti-integrin, are both approved for the treatment of CD and UC. Multiple anti-IL-23 agents, including guselkumab, are currently being evaluated in Phase 3 programs for both CD and UC. In addition, several oral small molecule therapies are currently being evaluated in both CD and UC, including Janus kinase (JAK) inhibitors and sphingosine-1-phosphate (S1P) receptor modulators.

With the availability of newer classes of agents, treatment targets have been evolving. It is becoming increasingly clear that treatments aimed purely at controlling symptoms do not alter the natural progression of IBD, as they often fail to repair the underlying inflammatory lesions (Bouguen G et al., Endoscopic assessment and treating to target increase the likelihood of mucosal healing in patients with Crohn's disease. Clin Gastroenterol Hepatol. 2014; 12 (6): 978-985; Colombel J F et al., Inflammatory Bowel Disease 2017: Innovations and Changing Paradigms. Gastroenterology. 2017; 152 (2): 309-312). Currently, IBD management has shifted towards a greater focus on preventing disease progression and improving long-term outcomes for patients (D'Haens 2014).

Despite the substantial advances conferred with advanced therapies as monotherapies, significant unmet need remains in the treatment of CD. Even with the best available approved therapies, more than half of patients with CD fail to achieve clinical remission after 1 year, with even fewer achieving endoscopic remission (Singh S et al., AGA Technical Review on the Medical Management of Moderate to Severe Luminal and Perianal Fistulizing Crohn's Disease. Gastroenterology. 2021; 160 (7): 2512-2556.e9). These statistics highlight the need for more effective therapies and treatment paradigms. Further, the efficacy plateau of monotherapies suggests a need for improved treatments that achieve higher rates of long-standing symptomatic and objective remission.

### SUMMARY OF INVENTION

One aspect of the invention is a method of treating Crohn's disease (CD) in a patient, the method comprising administering a combination of an IL-23 inhibitor and a TNF-α inhibitor, wherein the method results in a clinical response in the patient.

In one embodiment, the IL-23 inhibitor comprises an anti-IL-23p19 antibody or an antigen-binding fragment thereof and the TNF-α inhibitor comprises an anti-TNF-α antibody or an antigen-binding fragment thereof.

In one embodiment, the IL-23 inhibitor is selected from the group consisting of guselkumab, risankizumab, tildrakizumab and mirikizumab, and the TNF-α inhibitor is selected from the group consisting of golimumab, adalimumab, infliximab, certolizumab pegol and etanercept.

In one embodiment, the anti-IL-23p19 antibody comprises: a) heavy chain complementarity determining region (CDR) amino acid sequences of SEQ ID NOs: 1-3 and light chain CDR amino acid sequences of SEQ ID NOs: 4-6; b) a heavy chain variable region amino acid sequence of SEQ ID NO: 7 and a light chain variable region amino acid sequence of SEQ ID NO: 8; or c) a heavy chain amino acid sequence of SEQ ID NO: 9 and a light chain amino acid sequence of SEQ ID NO: 10.

In one embodiment, the anti-TNF-α antibody comprises: a) heavy chain CDR amino acid sequences of SEQ ID NOs: 11-13 and light chain CDR amino acid sequences of SEQ ID NOs: 14-16; b) a heavy chain variable region amino acid sequence of SEQ ID NO: 17 and a light chain variable region amino acid sequence of SEQ ID NO: 18; or c) a heavy chain amino acid sequence of SEQ ID NO: 19 and a light chain amino acid sequence of SEQ ID NO: 20.

In one embodiment, the anti-IL-23p19 antibody comprises: a) heavy chain CDR amino acid sequences of SEQ ID NOs: 1-3 and light chain CDR amino acid sequences of SEQ ID NOs: 4-6; b) a heavy chain variable region amino acid sequence of SEQ ID NO: 7 and a light chain variable region amino acid sequence of SEQ ID NO: 8; or c) a heavy chain amino acid sequence of SEQ ID NO: 9 and a light chain amino acid sequence of SEQ ID NO: 10, and the anti-TNF-α antibody comprises: a) heavy chain CDR amino acid sequences of SEQ ID NOs: 11-13 and light chain CDR amino acid sequences of SEQ ID NOs: 14-16; b) a heavy chain variable region amino acid sequence of SEQ ID NO: 17 and a light chain variable region amino acid sequence of SEQ ID NO: 18; or c) a heavy chain amino acid sequence of SEQ ID NO: 19 and a light chain amino acid sequence of SEQ ID NO: 20.

In one embodiment, the combination comprises the IL-23 inhibitor and the TNF-α inhibitor at a weight ratio of from about 2:1 to 1:2.

In one embodiment, the combination comprises the IL-23 inhibitor and the TNF-α inhibitor formulated in separate syringes and administered subcutaneously.

In one embodiment, i) the combination comprises the IL-23 inhibitor and the TNF-α inhibitor co-formulated in a single syringe and administered subcutaneously in a single administration or ii) the combination comprises the IL-23 inhibitor and the TNF-α inhibitor separately formulated in separate syringes and mixed and administered subcutaneously in a single administration.

In one embodiment, the combination comprises about 20-1000 mg of the IL-23 inhibitor and about 20-1000 mg of the TNF-α inhibitor and is administered subcutaneously every 1, 2, 3, 4, 5, 6, 7, or 8 weeks.

In one embodiment, the combination comprises about 320 mg of the IL-23 inhibitor and about 160 mg of the TNF-α inhibitor and is administered subcutaneously at weeks 0, 4, and 8.

In one embodiment, the combination comprises about 320 mg of the IL-23 inhibitor and about 160 mg of the TNF-α inhibitor and is administered subcutaneously at weeks 0, 4, and 8 and the method further comprises administering subcutaneously about 160 mg of the IL-23 inhibitor and about 80 mg of the TNF-α inhibitor every 4 weeks after week 8.

In one embodiment, combination comprises about 320 mg of the IL-23 inhibitor and about 160 mg of the TNF-α inhibitor and is administered subcutaneously at weeks 0, 4, and 8 and the method further comprises administering subcutaneously about 40 mg of the IL-23 inhibitor and about 40 mg of the TNF-α inhibitor every 4 weeks after week 8.

In one embodiment, the combination comprises about 160 mg of the IL-23 inhibitor and about 80 mg of the TNF-α inhibitor and is administered subcutaneously at weeks 0, 4, and 8.

In one embodiment, the combination comprises about 160 mg of the IL-23 inhibitor and about 80 mg of the TNF-α inhibitor and is administered subcutaneously at weeks 0, 4, and 8 and the method further comprises administering subcutaneously about 20 mg of the IL-23 inhibitor and about 20 mg of the TNF-α inhibitor every 4 weeks after week 8.

In one embodiment, the method comprises administering subcutaneously (i) about 160 mg of the IL-23 inhibitor and about 80 mg of the TNF-α inhibitor every 4 weeks, (ii) about 40 mg of the IL-23 inhibitor and about 40 mg of the TNF-α inhibitor every 4 weeks, or (iii) about 20 mg of the IL-23 inhibitor and about 20 mg of the TNF-α inhibitor every 4 weeks.

In one embodiment, the patient suffers moderately or severely active CD.

In one embodiment, the patient suffers moderately or severely active CD and was previously treated with ≥1 advanced therapy (ADT), and wherein the patient did not undergo remission or had an inadequate initial clinical response, loss of clinical response, or intolerance to the previous treatment. In such embodiment, the ADT agents may include, without limitation, TNFα antagonists, anti-IL23 medications, and other therapeutics classes such as natalizumab, ustekinumab, vedolizumab, rituximab, etc., as branded or as biosimilars.

In one embodiment, the patient suffers moderately or severely active CD and was previously treated with a TNF-α inhibitor alone and wherein the CD did not undergo remission after the previous treatment.

In one embodiment, the patient suffers moderately or severely active CD and was previously treated with an IL-23 inhibitor alone and wherein the CD did not undergo remission after the previous treatment.

In one embodiment, the patient suffers moderately or severely active CD and was previously treated with a TNF-α inhibitor or an IL-23 inhibitor alone, and wherein the patient had an inadequate initial clinical response, loss of clinical response, or intolerance to ≥1 previous ADT.

In one embodiment, the clinical response is based on a clinical endpoint selected from the group consisting of: (i) achievement of Crohn's Disease Activity Index (CDAI) score <150, (ii) achievement of a ≥50% improvement from baseline in Simple Endoscopic Score for Crohn's Disease (SES-CD) score, (iii) achievement of an abdominal pain (AP) mean daily score ≤1 and stool frequency (SF) mean daily score ≤3, and no worsening of the AP or SF from baseline, and (iv) achievement of SES-CD≤2 or SES-CD score ≤4 with at least 2 points reduction from baseline and no subscore >1 in any individual component.

In one embodiment, the clinical response is measured about 24 weeks, 48 weeks, or 240 weeks after initial treatment.

In one embodiment, the method is clinically safe in treating the patient, or wherein the method results in a reduced adverse effect compared to a treatment with a TNF-α inhibitor alone or an IL-23 inhibitor alone.

Another aspect of the invention is a kit comprising (1) an IL-23 inhibitor and a TNF-α inhibitor, and (2) instructions for treating CD in a patient, wherein the instructions comprise subcutaneously administering to the patient (i) about 320 mg of the IL-23 inhibitor and about 160 mg of the TNF-α inhibitor at weeks 0, 4, and 8; (ii) about 160 mg of the IL-23 inhibitor and about 80 mg of the TNF-α inhibitor at weeks 0, 4, and 8; (iii) about 160 mg of the IL-23 inhibitor and about 80 mg of the TNF-α inhibitor every 4 weeks; (iv) about 40 mg of the IL-23 inhibitor and about 40 mg of the TNF-α inhibitor every 4 weeks; or (v) about 20 mg of the IL-23 inhibitor and about 20 mg of the TNF-α inhibitor every 4 weeks.

Another aspect of the invention is a method of treating CD in a patent, the method comprising administering a combination of an anti-IL-23p19 antibody and an anti-TNF-α antibody, wherein,
a) the anti-IL-23p19 antibody comprises (i) the heavy chain CDR amino acid sequences of SEQ ID NOs: 1-3 and the light chain CDR amino acid sequences of SEQ ID NOs: 4-6, (ii) the heavy chain variable region amino acid sequence of SEQ ID NO: 7 and the light chain variable region amino acid sequence of SEQ ID NO: 8, or (iii) the heavy chain amino acid sequence of SEQ ID NO: 9 and the light chain amino acid sequence of SEQ ID NO: 10;
b) the anti-TNF-α antibody comprises (i) the heavy chain CDR amino acid sequences of SEQ ID NOs: 11-13 and the light chain CDR amino acid sequences of SEQ ID NOs: 14-16, (ii) the heavy chain variable region amino acid sequence of SEQ ID NO: 17 and the light chain variable region amino acid sequence of SEQ ID NO: 18, or (iii) the heavy chain amino acid sequence of SEQ ID NO: 19 and the light chain amino acid sequence of SEQ ID NO: 20; and
c) the method results in a clinical response in the patient and the clinical response is based on a clinical endpoint selected from the group consisting of: (i) achievement of Crohn's Disease Activity Index (CDAI) score <150, (ii) achievement of a ≥50% improvement from baseline in Simple Endoscopic Score for Crohn's Disease (SES-CD) score, (iii) achievement of an abdominal pain (AP) mean daily score ≤1 and stool frequency (SF) mean daily score ≤3, and no worsening of the AP or SF from baseline, and (iv) achievement of SES-CD≤2 or SES-CD score ≤4 with at least 2 points reduction from baseline and no subscore >1 in any individual component.

In one embodiment, the combination comprises the anti-IL-23p19 antibody and the anti-TNF-α antibody at a weight ratio of from about 2:1 to 1:2.

In one embodiment, the combination comprises the anti-IL-23p19 antibody and the anti-TNF-α antibody formulated in separate syringes and administered subcutaneously.

In one embodiment, i) the combination comprises the anti-IL-23p19 antibody and the anti-TNF-α antibody co-formulated in a single syringe and administered subcutaneously in a single administration or ii) the combination comprises the anti-IL-23p19 antibody and the anti-TNF-α antibody separately formulated in separate syringes and mixed and administered subcutaneously in a single administration.

In one embodiment, the combination comprises about 20-1000 mg of the anti-IL-23 antibody and about 20-1000 mg of the anti-TNF-α antibody and is administered subcutaneously every 1, 2, 3, 4, 5, 6, 7, or 8 weeks.

In one embodiment, the combination comprises about 320 mg of the anti-IL-23 antibody and about 160 mg of the anti-TNF-α antibody and is administered subcutaneously at weeks 0, 4, and 8.

In one embodiment, the combination comprises about 320 mg of the anti-IL-23 antibody and about 160 mg of the anti-TNF-α antibody and is administered subcutaneously at weeks 0, 4, and 8 and the method further comprises administering subcutaneously about 160 mg of the anti-IL-23p19 antibody and about 80 mg of the anti-TNF-α antibody every 4 weeks after week 8.

In one embodiment, the combination comprises about 320 mg of the anti-IL-23 antibody and about 160 mg of the anti-TNF-α antibody and is administered subcutaneously at weeks 0, 4, and 8 and the method further comprises administering subcutaneously about 40 mg of the anti-IL-23p19 antibody and about 40 mg of the anti-TNF-α antibody every 4 weeks after week 8.

In one embodiment, the combination comprises about 160 mg of the anti-IL-23p19 antibody and about 80 mg of the anti-TNF-α antibody and is administered subcutaneously at weeks 0, 4, and 8.

In one embodiment, the combination comprises about 160 mg of the anti-IL-23p19 antibody and about 80 mg of the anti-TNF-α antibody and is administered subcutaneously at weeks 0, 4, and 8 and the method further comprises administering subcutaneously about 20 mg of the anti-IL-23 antibody and about 20 mg of the anti-TNF-α antibody every 4 weeks after week 8.

In one embodiment, the method comprises administering subcutaneously (i) about 160 mg of the anti-IL-23p19 antibody and about 80 mg of the anti-TNF-α antibody every 4 weeks, (ii) about 40 mg of the anti-IL-23p19 antibody and about 40 mg of the anti-TNF-α antibody every 4 weeks, or (iii) about 20 mg of the anti-IL-23p19 antibody and about 20 mg of the anti-TNF-α antibody every 4 weeks.

In one embodiment, the patient suffers moderately or severely active CD.

In some embodiment, the patient suffers moderately or severely active CD and was previously treated with ≥1 ADT, and wherein the patient did not undergo remission or had an inadequate initial clinical response, loss of clinical response, or intolerance to the previous treatment. For example, in one embodiment, the patient suffers moderately or severely active CD and was previously treated with a TNF-α inhibitor alone and wherein the CD did not undergo remission after the previous treatment.

In one embodiment, the patient suffers moderately or severely active CD and was previously treated with an IL-23 inhibitor alone and wherein the CD did not undergo remission after the previous treatment.

In one embodiment, the patient suffers moderately or severely active CD and was previously treated with a TNF-α inhibitor alone and wherein the CD did not undergo remission after the previous treatment.

In one embodiment, the patient suffers moderately or severely active CD and was previously treated with a TNF-α inhibitor or an IL-23 inhibitor alone, and wherein the patient had an inadequate initial clinical response, loss of clinical response, or intolerance to ≥1 previous ADT.

In one embodiment, the method is clinically safe in treating the patient, or wherein the method results in a reduced adverse effect compared to a treatment with a TNF-α inhibitor alone or an IL-23 inhibitor alone.

### DETAILED DESCRIPTION

### Definitions

Unless defined otherwise, technical and scientific terms used herein have the same meaning as commonly understood by one of ordinary skill in the art to which this invention belongs.

As used herein, including the appended claims, the singular forms of words such as “a,” “an,” and “the,” include their corresponding plural references unless the context clearly dictates otherwise.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell.

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts a target, such as IL-23 receptor, e.g., in the fluid phase or colloidal phase, but also situations where the agonist or antagonist does not contact the cell or the receptor.

“Treat” or “treating” may also refer to administration of a therapeutic agent, such as a composition described herein, internally or externally to a patient in need of the therapeutic agent. Typically, the agent is administered in an amount effective to prevent or alleviate one or more disease symptoms, or one or more adverse effects of treatment with a different therapeutic agent, whether by preventing the development of, inducing the regression of, or inhibiting the progression of such symptom(s) or adverse effect(s) by any clinically measurable degree. The amount of a therapeutic agent that is effective to alleviate any particular disease symptom or adverse effect (also referred to as the “therapeutically effective amount”) may vary according to factors such as the disease state, age, and weight of the patient, the ability of the therapeutic agent to elicit a desired response in the patient, the overall health of the patient, the method, route and dose of administration, and the severity of side effects.

An “inhibitor,” as used herein, is any agent that reduces the activity of a targeted molecule. Specifically, an antagonist of IL-23 or TNF-α is an agent that reduces the biological activity of IL-23 or TNF-α, for example by blocking binding of IL-23 or TNF-α to its receptor or otherwise reducing its activity (e.g. as measured in a bioassay).

Interleukin IL-23 is a heterodimer composed of two subunits: IL-23A (p19) and IL-12B (p40). It has about 60 kDa. The genes for the two subunits of human IL-23 are differently located: the IL23A gene (coding for p19) is on chromosome 5q31-33, whereas the IL12B gene (encoding p40) is on chromosome 12q13.

As used herein, an “anti-IL-23 specific antibody,” “anti-IL-23 antibody,” “antibody portion,” or “antibody fragment” and/or “antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-23 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-23 activity or binding, or with IL-23 receptor activity or binding, in vitro, in situ and/or in vivo. As a non-limiting example, a suitable anti-IL-23 antibody, specified portion or variant of the present invention can bind at least one IL-23 molecule, or specified portions, variants or domains thereof. A suitable anti-IL-23 antibody, specified portion, or variant can also optionally affect at least one of IL-23 activity or function, such as but not limited to, RNA, DNA or protein synthesis, IL-23 release, IL-23 receptor signaling, membrane IL-23 cleavage, IL-23 activity, IL-23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-23. For example, antibody fragments capable of binding to IL-23 or portions thereof, include, but are not limited to, Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments.

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin. If the antibody contains a constant region or a portion of the constant region, the constant region also is derived from sequences of human origin.

“Subject” or “patient” as used interchangeably includes any human or nonhuman animal.

“Nonhuman animal” includes all vertebrates, e.g., mammals and non-mammals, such as nonhuman primates, sheep, dogs, cats, horses, cows, chickens, amphibians, reptiles, etc.

“Tumor necrosis factor,” “TNF” or “TNF-α” refers to the well-known human tumor necrosis factor-α (TNF-α), a multifunctional pro-inflammatory cytokine. TNF-α triggers pro-inflammatory pathways that result in tissue injury, such as degradation of cartilage and bone, induction of adhesion molecules, induction of pro-coagulant activity on vascular endothelial cells, an increase in the adherence of neutrophils and lymphocytes, and stimulation of the release of platelet activating factor from macrophages, neutrophils and vascular endothelial cells. TNF-α is found as a soluble protein as well as a precursor form called transmembrane TNF-α that is expressed as a cell surface type II polypeptide. Transmembrane TNF-α is processed by metalloproteinases such as TNF-α-converting enzyme (TACE) between residues Ala76 and Val77, resulting in the release of the soluble form of TNF-α of 157 amino acid residues. Soluble TNF-α is a homotrimer of 17-kDa cleaved monomers. Transmembrane TNF-α also exists as a homotrimer of 26-kD uncleaved monomers.

In a first aspect is provided a method of treating Crohn's disease (CD) in a subject (e.g., a human patient). The method comprises administering a combination of an IL-23 inhibitor and a TNF-α inhibitor. The method is effective and safe to treat the CD.

Various IL-23 inhibitors may be used herein. In one embodiment, the IL-23 inhibitor is selected from anti-IL-23 antibodies or antigen-binding fragments thereof, such as antibodies or antigen-binding fragments thereof that target or bind to the p19 subunit of IL-23 (i.e., anti-IL-23p19 antibody).

Various TNF-α inhibitors may be used herein. In one embodiment, the TNF-α inhibitor is selected from anti-TNF-α antibodies or antigen-binding fragments thereof that target or bind to TNF-α.

Various host animals may be used to produce anti-IL-23 antibodies (e.g., anti-IL-23p19 antibodies) and anti-TNF-α antibodies. For example, Balb/c mice may be used to generate mouse anti-human IL-23 antibodies or mouse anti-human TNF-α antibodies. The antibodies made in Balb/c mice and other non-human animals may be humanized using various technologies to generate more human-like sequences.

Anti-IL-23 antibodies can optionally be characterized by high affinity binding to IL-23 and, optionally, having low toxicity. Anti-TNF-α antibodies can optionally be characterized by high affinity binding to TNF-α and, optionally, having low toxicity. In particular, an antibody, specified fragment or variant of the antibody may be used in where the individual components, such as the variable region, constant region and framework, individually and/or collectively, optionally and preferably possess low immunogenicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titers in the patient treated (less than about 300, preferably less than about 100 measured with a double antigen enzyme immunoassay) (Elliott et al., Lancet 344:1125-1127 (1994), entirely incorporated herein by reference). For anti-IL-23 antibodies, “low immunogenicity” can also be defined as the incidence of titratable levels of antibodies to the anti-IL-23 antibody in patients treated with anti-IL-23 antibody as occurring in less than 25% of patients treated, preferably, in less than 10% of patients treated with the recommended dose for the recommended course of therapy during the treatment period. For the anti-TNF-α antibodies, “low immunogenicity” can also be defined as the incidence of titratable levels of antibodies to the anti-TNF-α antibody in patients treated with anti-TNF-α antibody as occurring in less than 25% of patients treated, preferably, in less than 10% of patients treated with the recommended dose for the recommended course of therapy during the treatment period.

The anti-IL-23 antibodies and anti-TNF-α antibodies used in the methods described herein may be produced by a cell line, a mixed cell line, an immortalized cell or clonal population of immortalized cells, as well known in the art. See, e.g., Ausubel, et al., ed., Current Protocols Molecular Biology, John Wiley & Sons, Inc., NY (1987-2001); Sambrook, et al., Molecular Cloning: A Laboratory Manual, 2nd Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY (1997-2001), each entirely incorporated herein by reference herein.

The anti-IL-23 antibodies and/or anti-TNF-α antibodies can also be generated by immunization of a transgenic animal (e.g., mouse, rat, hamster, non-human primate, and the like) capable of producing a repertoire of human antibodies, as described herein and/or as known in the art. Cells that produce a human anti-IL-23 antibody or human anti-TNF-α antibody can be isolated from such animals and immortalized using suitable methods, such as the methods described herein.

The anti-IL-23 antibodies used in the methods described herein can also be prepared using an anti-IL-23 antibody encoding nucleic acid to provide transgenic animals or mammals, such as goats, cows, horses, sheep, rabbits, and the like, that produce such antibodies in their milk. The anti-TNF-α antibodies used in the methods described herein can also be prepared using an anti-TNF-α antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

The anti-IL-23 antibodies can bind human IL-23 with a wide range of affinities (KD). In one embodiment, a human mAb can optionally bind human IL-23 with high affinity. For example, a human mAb can bind human IL-23 with a KD equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The anti-TNF-α antibodies can bind human TNF-α with a wide range of affinities (KD). In one embodiment, a human mAb can optionally bind human TNF-α with high affinity. For example, a human mAb can bind human TNF-α with a KD equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The anti-IL-23 antibodies may be an IgG1, IgG2, IgG3 or IgG4 isotype. The anti-TNF-α antibodies may be an IgG1, IgG2, IgG3 or IgG4 isotype.

Anti-IL-23 antibodies and/or anti-TNFα antibodies can also be humanized or prepared as human antibodies engineered with retention of high affinity for the antigen and other favorable biological properties. Humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

Humanization or engineering of antibodies used herein can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151:2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), and U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference.

In one embodiment, the IL-23 inhibitor used herein is selected from anti-IL-23 antibodies or antigen-binding fragments thereof, which include, without limitation, guselkumab, risankizumab, tildrakizumab and mirikizumab. In one embodiment, the IL-23 inhibitor is selected from any of the anti-IL-23p19 antibodies and antigen-binding fragments thereof described in U.S. Pat. No. 7,491,391 and U.S. Patent Application Publication No. 2018/0094052, the entire disclosure of which are incorporated herein by reference.

In one embodiment, the IL-23 inhibitor used herein is an anti-IL-23p19 antibody or an antigen-binding fragment thereof comprising complementarity determining region (CDR) sequences of: (i) heavy chain CDR amino acid sequences of SEQ ID NO: 1 (CDRH1), SEQ ID NO: 2 (CDRH2), and SEQ ID NO: 3 (CDRH3); and (ii) light chain CDR amino acid sequences of SEQ ID NO: 4 (CDRL1), SEQ ID NO: 5 (CDRL2), and SEQ ID NO: 6 (CDRL3).

In one embodiment, the IL-23 inhibitor used herein is an anti-IL-23p19 antibody or an antigen-binding fragment thereof comprising a heavy chain variable region amino acid sequence of SEQ ID NO: 7 and a light chain variable region amino acid sequence of SEQ ID NO: 8.

In one embodiment, the IL-23 inhibitor used herein is an anti-IL-23p19 antibody or an antigen-binding fragment thereof comprising a heavy chain amino acid sequence of SEQ ID NO: 9 and a light chain amino acid sequence of SEQ ID NO: 10.

TABLE 1
Anti-IL23p19 Antibody Sequences:

| SEQ ID | | |
|---|---|---|
| NO: | Description | Sequence |
| 1 | HCDR1 | NYWIG |
| 2 | HCDR2 | IIDPSNSYTR YSPSFQG |
| 3 | HCDR3 | WYYKPFDV |
| 4 | LCDR1 | TGSSSNIGSG YDVH |
| 5 | LCDR2 | GNSKRPS |
| 6 | LCDR3 | ASWTDGLSLV V |
| 7 | VH | EVQLVQSGAE VKKPGESLKI SCKGSGYSFS NYWIGWVRQM |
| | | PGKGLEWMGI IDPSNSYTRY SPSFQGQVTI SADKSISTAY |
| | | LQWSSLKASD TAMYYCARWY YKPFDVWGQG TLVTVSS |
| 8 | VL | QSVLTQPPSV SGAPGQRVTI SCTGSSSNIG SGYDVHWYQQ |
| | | LPGTAPKLLI YGNSKRPSGV PDRFSGSKSG TSASLAITGL |
| | | QSEDEADYYC ASWTDGLSLV VFGGGTKLTV L |
| 9 | Heavy Chain | EVQLVQSGAE VKKPGESLKI SCKGSGYSFS NYWIGWVRQM |
| | | PGKGLEWMGI IDPSNSYTRY SPSFQGQVTI SADKSISTAY |
| | | LQWSSLKASD TAMYYCARWY YKPFDVWGQG TLVTVSSAST |
| | | KGPSVFPLAP SSKSTSGGTA ALGCLVKDYF PEPVTVSWNS |
| | | GALTSGVHTF PAVLQSSGLY SLSSVVTVPS SSLGTQTYIC |
| | | NVNHKPSNTK VDKKVEPKSC DKTHTCPPCP APELLGGPSV |
| | | FLFPPKPKDT LMISRTPEVT CVVVDVSHED PEVKFNWYVD |
| | | GVEVHNAKTK PREEQYNSTY RVVSVLTVLH QDWLNGKEYK |
| | | CKVSNKALPA PIEKTISKAK GQPREPQVYT LPPSRDELTK |
| | | NQVSLTCLVK GFYPSDIAVE WESNGQPENN YKTTPPVLDS |
| | | DGSFFLYSKL TVDKSRWQQG NVFSCSVMHE ALHNHYTQKS |
| | | LSLSPGK |
| 10 | Light Chain | QSVLTQPPSV SGAPGQRVTI SCTGSSSNIG SGYDVHWYQQ |
| | | LPGTAPKLLI YGNSKRPSGV PDRFSGSKSG TSASLAITGL |
| | | QSEDEADYYC ASWTDGLSLV VFGGGTKLTV LGQPKAAPSV |
| | | TLFPPSSEEL QANKATLVCL ISDFYPGAVT VAWKADSSPV |
| | | KAGVETTTPS KQSNNKYAAS SYLSLTPEQW KSHRSYSCQV |
| | | THEGSTVEKT VAPTECS |

In one embodiment, the IL-23 inhibitor used herein is guselkumab (an anti-IL-23p19 antibody marketed by Janssen Biotech, Inc. under the tradename TREMFYA®)

In one embodiment, the TNF-α inhibitor used herein is selected from golimumab, adalimumab, infliximab, certolizumab pegol, and etanercept. In one embodiment, the TNF-α inhibitor is selected from the anti-TNF-α antibodies and antigen-binding fragments thereof described in U.S. Pat. No. 7,250,165 and U.S. Patent Application Publication No. 2017/0218092, the entire disclosure of which are incorporated herein by reference.

In one embodiment, the TNF-α inhibitor used herein is an anti-TNF-α antibody or an antigen-binding fragment thereof comprising CDR sequences of: (i) heavy chain CDR amino acid sequences of SEQ ID NO: 11 (CDRH1), SEQ ID NO: 12 (CDRH2), and SEQ ID NO: 13 (CDRH3); and (ii) light chain CDR amino acid sequences of SEQ ID NO: 14 (CDRL1), SEQ ID NO: 15 (CDRL2), and SEQ ID NO: 16 (CDRL3).

In one embodiment, the TNF-α inhibitor used herein is an anti-TNF-α antibody or an antigen-binding fragment thereof comprising a heavy chain variable region amino acid sequence of SEQ ID NO: 17 and a light chain variable region amino acid sequence of SEQ ID NO: 18.

In one embodiment, the TNF-α inhibitor used herein is an anti-TNF-α antibody or an antigen-binding fragment thereof comprising a heavy chain amino acid sequence of SEQ ID NO: 19 and a light chain amino acid sequence of SEQ ID NO: 20.

TABLE 2
Anti-TNF-α Antibody Sequences

| SEQ ID | | |
|---|---|---|
| NO: | Description | Sequence |
| 11 | HCDR1 | SYAMH |
| 12 | HCDR2 | FMSYDGSNKK YADSVKG |
| 13 | HCDR3 | DRGIAAGGNY YYYGMDV |
| 14 | LCDR1 | RASQSVYSYL A |
| 15 | LCDR2 | DASNRAT |
| 16 | LCDR3 | QQRSNWPPFT |
| 17 | VH | QVQLVESGGG VVQPGRSLRL SCAASGFIFS SYAMHWVRQA |
| | | PGNGLEWVAF MSYDGSNKKY ADSVKGRFTI SRDNSKNTLY |
| | | LQMNSLRAED TAVYYCARDR GIAAGGNYYY YGMDVWGQGT |
| | | TVTVSS |
| 18 | VL | EIVLTQSPAT LSLSPGERAT LSCRASQSVY SYLAWYQQKP |
| | | GQAPRLLIYD ASNRATGIPA RFSGSGSGTD FTLTISSLEP |
| | | EDFAVYYCQQ RSNWPPFTFG PGTKVDIKRT V |
| 19 | Heavy Chain | QVQLVESGGG VVQPGRSLRL SCAASGFIFS SYAMHWVRQA |
| | | PGNGLEWVAF MSYDGSNKKY ADSVKGRFTI SRDNSKNTLY |
| | | LQMNSLRAED TAVYYCARDR GIAAGGNYYY YGMDVWGQGT |
| | | TVTVSSASTK GPSVFPLAPS SKSTSGGTAA LGCLVKDYFP |
| | | EPVTVSWNSG ALTSGVHTFP AVLQSSGLYS LSSVVTVPSS |
| | | SLGTQTYICN VNHKPSNTKV DKKVEPKSCD KTHTCPPCPA |
| | | PELLGGPSVF LFPPKPKDTL MISRTPEVTC VVVDVSHEDP |
| | | EVKFNWYVDG VEVHNAKTKP REEQYNSTYR VVSVLTVLHQ |
| | | DWLNGKEYKC KVSNKALPAP IEKTISKAKG QPREPQVYTL |
| | | PPSRDELTKN QVSLTCLVKG FYPSDIAVEW ESNGQPENNY |
| | | KTTPPVLDSD GSFFLYSKLT VDKSRWQQGN VFSCSVMHEA |
| | | LHNHYTQKSL SLSPGK |
| 20 | Light Chain | EIVLTQSPAT LSLSPGERAT LSCRASQSVY SYLAWYQQKP |
| | | GQAPRLLIYD ASNRATGIPA RFSGSGSGTD FTLTISSLEP |
| | | EDFAVYYCQQ RSNWPPFTFG PGTKVDIKRT VAAPSVFIFP |
| | | PSDEQLKSGT ASVVCLLNNF YPREAKVQWK VDNALQSGNS |
| | | QESVTEQDSK DSTYSLSSTL TLSKADYEKH KVYACEVTHQ |
| | | GLSSPVTKSF NRGEC |

In one embodiment, the TNF-α inhibitor used herein is golimumab (an anti-TNF-α antibody marketed by Janssen Biotech, Inc. under the tradename SIMPONI®)

Administering IL-23 inhibitor and TNF-α inhibitor in subjects suffering from inflammatory bowel disease (IBD) including CD is disclosed in PCT Patent Application Publication No. WO2020/234834; PCT Patent Application No. PCT/IB2021/054390; and U.S. Patent Application No. 63/191,076, the entire disclosure of each of the above are incorporated herein by reference.

In one embodiment, the method of administering a combination of the IL-23 inhibitor and the TNF-α inhibitor can result in a clinical response in the subject suffering from CD. In one embodiment, the subject may be suffering from moderately or severely active CD. CD may be confined to the colon, but may also be present in other locations within the gastrointestinal tract such as the small intestine. CD can involve inflammation of the colon and small intestine. There may even be inflammation of the mouth, anus, skin, eyes, joints, and/or liver. The clinical response may be based on a clinical endpoint selected from: (i) achievement of Crohn's Disease Activity Index (CDAI) score <150, (ii) achievement of a ≥50% improvement from baseline in Simple Endoscopic Score for Crohn's Disease (SES-CD) score, (iii) achievement of an abdominal pain (AP) mean daily score ≤1 and stool frequency (SF) mean daily score ≤3, and no worsening of the AP or SF from baseline, and (iv) achievement of SES-CD≤2 or SES-CD score ≤4 with at least 2 points reduction from baseline and no subscore >1 in any individual component. The clinical response may be measured about 8 or more weeks after the initial treatment. In one embodiment, the clinical response may be measured about 24 weeks, 48 weeks, or 240 weeks after the initial treatment.

In some embodiment, the subject was previously treated with ≥1 ADT as disclosed herein. For example, in one embodiment, the subject was previously treated with a TNF-α inhibitor (such as an anti-TNF-α antibody) alone and the CD did not undergo remission after the previous treatment. In one embodiment, the patient was previously treated with an IL-23 inhibitor (such as an anti-IL-23p19 antibody) alone and the CD did not undergo remission after the previous treatment. In one embodiment, the patient was previously treated with a TNF-α inhibitor (such as an anti-TNF-α antibody) or an IL-23 inhibitor (such as an anti-IL-23p19 antibody) alone and the patient had an inadequate initial clinical response, loss of clinical response, or intolerance to ≥1 previous ADT. The methods described herein may be beneficial for subjects who did not respond to monotherapy treatments with either IL-23 inhibitor (e.g., an anti-IL-23p19 antibody) or TNF-α inhibitor (e.g., an anti-TNF-α antibody). The methods described herein is clinically safe. The methods described herein may result in a reduced adverse effect compared to monotherapy treatments with either IL-23 inhibitor (e.g., an anti-IL-23p19 antibody) or TNF-α inhibitor (e.g., an anti-TNF-α antibody) alone.

Without wishing to be bound by theory, the benefits of combining an IL-23 inhibitor (e.g., an anti-IL-23p19 antibody) with a TNF-α inhibitor (e.g., an anti-TNF-α antibody) can arise from distinct gene expression changes induced by each antibody. As it has been demonstrated (see e.g., PCT Patent Application Publication No. WO2020/234834), at doses where each antibody provided similar protection against colonic inflammation, distinct intestinal gene expression changes were observed in mice when blocking IL-23p19 compared to blocking TNF-α. These gene expression changes may apply to human disease as well. Integration of ‘humanized’ murine anti-TNF-α and anti-IL-23p19 gene signatures with a human intestinal biopsy gene network can allow for focus only on genes that were expressed and varied in human intestinal tissues. Additional context for the potential molecular impact of each antibody on human IBD, including CD, can be obtained by generating treatment subnetworks that included genes one step removed in the network (i.e. strongly correlated) from genes within each signature. Individual anti-TNFα and anti-IL-23p19 subnetworks show unique single antibody gene signatures, allowing for insight into the biology targeted by both mechanisms.

In one embodiment, the IL-23 inhibitor (e.g., an anti-IL-23p19 antibody) and the TNF-α inhibitor (e.g., an anti-TNF-α antibody) are administered in a ratio of from about 2:1 to 1:2 (w/w). The ratio may be calculated from the dosage of one antibody in a subject in mg/kg and the dosage of the other antibody in the same subject in mg/kg.

Administration to a subject of an IL-23 inhibitor (e.g., an anti-TNF-α antibody) and a TNF-α inhibitor (e.g., an anti-IL-23p19 antibody) in a ratio of from about 2:1 to 1:2 (w/w) can provide for enhanced treatment of CD in the subject. In some embodiments, the ratio of the IL-23 inhibitor and the TNF-α inhibitor is from about 2:1 to 1.8:1 (w/w). In some embodiments, the ratio of the IL-23 inhibitor to the TNF-α inhibitor is from about 1.9:1 to 1.7:1 (w/w). In some embodiments, the ratio of the IL-23 inhibitor to the TNF-α inhibitor is from about 1.8:1 to 1.6:1 (w/w). In some embodiments, the ratio of the IL-23 inhibitor to the TNF-α inhibitor is from about 1.7:1 to 1.5:1 (w/w). In some embodiments, the ratio of the IL-23 inhibitor to the TNF-α inhibitor is from about 1.6:1 to 1.4:1 (w/w). In some embodiments, the ratio of the IL-23 inhibitor to the TNF-α inhibitor is from about 1.5:1 to 1.3:1 (w/w). In some embodiments, the ratio of the IL-23 inhibitor to the TNF-α inhibitor is from about 1.4:1 to 1.2:1 (w/w). In some embodiments, the ratio of the IL-23 inhibitor to the TNF-α inhibitor is from about 1.3:1 to 1.1:1 (w/w). In some embodiments, the ratio of the IL-23 inhibitor to the TNF-α inhibitor is from about 1.2:1 to 1:1 (w/w). In some embodiments, the ratio of the IL-23 inhibitor to the TNF-α inhibitor is from about 1.1:1 to 1:1 (w/w). In some embodiments, the ratio of the IL-23 inhibitor to the TNF-α inhibitor is from about 1:1 to 1:1.2 (w/w). In some embodiments, the ratio of the IL-23 inhibitor to the TNF-α inhibitor is from about 1:1.1 to 1:1.3 (w/w). In some embodiments, the ratio of the IL-23 inhibitor to the TNF-α inhibitor is from about 1:1.2 to 1:1.4 (w/w). In some embodiments, the ratio of the IL-23 inhibitor to the TNF-α inhibitor is from about 1:1.3 to 1:1.5 (w/w). In some embodiments, the ratio of the IL-23 inhibitor to the TNF-α inhibitor is from about 1:1.4 to 1:1.6 (w/w). In some embodiments, the ratio of the IL-23 inhibitor to the TNF-α inhibitor is from about 1:1.5 to 1:1.7 (w/w). In some embodiments, the ratio of the IL-23 inhibitor to the TNF-α inhibitor is from about 1:1.6 to 1:1.8 (w/w). In some embodiments, the ratio of the IL-23 inhibitor to the TNF-α inhibitor is from about 1:1.7 to 1:1.9 (w/w). In some embodiments, the ratio of the IL-23 inhibitor to the TNF-α inhibitor is from about 1:1.8 to 1:2 (w/w). In some embodiments, the ratio of the IL-23 inhibitor to the TNF-α inhibitor is about 2:1, 1.8:1, 1.5:1, 1.2:1, 1:1, 1:1.2, 1:1.5, 1:1.8 or 1:2 (w/w).

In one embodiment, the combination of the IL-23 inhibitor (e.g., an anti-IL-23p19 antibody) and the TNF-α inhibitor (e.g, an anti-TNF-α antibody) may be administered simultaneously, sequentially, or within one day of one another. In one embodiment, the combination is administered in one single administration as in Example 1.

The combination of the IL-23 inhibitor and the TNF-α inhibitor may be administered intravenously or subcutaneously daily; every two days; every three days; every four days, every five days, every six days, or once every 1, 2, 3, 4, 5, 6, 7, or 8 weeks. In one embodiment, the combination of about 20-1000 mg of the IL-23 inhibitor (e.g., an anti-IL-23p19 antibody) and about 20-1000 mg of the TNF-α inhibitor (e.g., an anti-TNF-α antibody) may be administered subcutaneously every 1, 2, 3, 4, 5, 6, 7, or 8 weeks.

In one embodiment, a combination of about 320 mg of the IL-23 inhibitor (e.g., an anti-IL-23p19 antibody) and about 160 mg of the TNF-α inhibitor (e.g., an anti-TNF-α antibody), or a combination of about 160 mg of the IL-23 inhibitor and about 80 mg of the TNF-α inhibitor, or a combination of about 40 mg of the IL-23 inhibitor and about 40 mg of the TNF-α inhibitor is administered subcutaneously every 4 weeks, or a combination of about 20 mg of the IL-23 inhibitor and about 20 mg of the TNF-α inhibitor is administered subcutaneously every 4 weeks.

In one embodiment, the method comprises an induction dosing period followed by a maintenance dosing period, wherein a combination of about 320 mg of the IL-23 inhibitor and about 160 mg of the TNF-α inhibitor is administered subcutaneously at weeks 0, 4, and 8 during the induction dosing period and a combination of about 160 mg of the IL-23 inhibitor and about 80 mg of the TNF-α inhibitor is administered subcutaneously every 4 weeks after week 8 during the maintenance dosing period. In one embodiment, a combination of about 320 mg of the IL-23 inhibitor and about 160 mg of the TNF-α inhibitor is administered subcutaneously at weeks 0, 4, and 8 during the induction dosing period and a combination of about 40 mg of the IL-23 inhibitor and about 40 mg of the TNF-α inhibitor is administered subcutaneously every 4 weeks after week 8 during the maintenance dosing period. In one embodiment, a combination of about 160 mg of the IL-23 inhibitor and about 80 mg of the TNF-α inhibitor is administered subcutaneously at weeks 0, 4, and 8 during the induction dosing period and a combination of about 20 mg of the IL-23 inhibitor and about 20 mg of the TNF-α inhibitor is administered subcutaneously every 4 weeks after week 8 during the maintenance dosing period.

The IL-23 inhibitor (e.g., an anti-IL-23p19 antibody) and the TNF-α inhibitor (e.g., and anti-TNF-α antibody) may be formulated separately or co-formulated together in stable formulations. The stable formulations may comprise a phosphate buffer with saline or a chosen salt, as well as preserved solutions and formulations containing a preservative as well as multi-use preserved formulations suitable for pharmaceutical or veterinary use, comprising an IL-23 inhibitor and/or a TNF-α inhibitor in a pharmaceutically acceptable formulation. Preserved formulations may contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, polymers, or mixtures thereof in an aqueous diluent. Any suitable concentration or mixture can be used, such as about 0.0015%, or any range, value, or fraction therein. Non-limiting examples include, without preservative, about 0.1-2% m-cresol (e.g., 0.2, 0.3, 0.4, 0.5, 0.9, 1.0%), about 0.1-3% benzyl alcohol (e.g., 0.5, 0.9, 1.1, 1.5, 1.9, 2.0, 2.5%), about 0.001-0.5% thimerosal (e.g., 0.005, 0.01), about 0.001-2.0% phenol (e.g., 0.05, 0.25, 0.28, 0.5, 0.9, 1.0%), about 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

The aqueous diluent may further comprise a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can be added to the formulations or compositions to reduce aggregation. These additives may be useful if a pump or plastic container is used to administer the formulation. The presence of pharmaceutically acceptable surfactant can reduce any propensity for an antibody to aggregate.

The formulations used herein can be prepared by a process that comprises mixing the IL-23 inhibitor and/or the TNF-α inhibitor with a selected buffer. The buffer can be a phosphate buffer containing saline or a chosen salt. Mixing the IL-23 inhibitor and/or the TNF-α inhibitor and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

Stable or preserved formulations comprising one or both of the IL-23 inhibitor and the TNF-α inhibitor can be provided to patients as clear solutions or as dual vials comprising a vial of lyophilized powder of one or both of the IL-23 inhibitor and the TNF-α inhibitor, which is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

For parenteral administration, the IL-23 inhibitor (e.g., the anti-IL-23p19 antibody) and/or the TNF-α inhibitor (e.g., the anti-TNF-α antibody) can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and about 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Many known and developed modes can be used herein for administering a combination of the IL-23 inhibitor (e.g., the anti-IL-23p19 antibody) and the TNF-α inhibitor (e.g., the anti-TNF-α antibody).

In one embodiment, the combination comprises the IL-23 inhibitor (e.g., an anti-IL-23p19 antibody) and the TNF-α inhibitor (e.g., an anti-TNF-α antibody) co-formulated at a desired ratio and administered in a single administration.

In one embodiment, the IL-23 inhibitor (e.g., an anti-IL-23p19 antibody) and the TNF-α inhibitor (e.g., an anti-TNF-α antibody) are separately formulated and administered simultaneously or sequentially, preferably within a same day. Or, the separately formulated IL-23 inhibitor and the TNF-α inhibitor may be mixed in desired ratio and then administered in a single administration.

For example, the IL-23 inhibitor used herein may be an anti-IL-23p19 antibody formulated in an aqueous solution at 100 mg/mL: 7.9% (w/v) sucrose, 4.0 mM Histidine, 6.9 mM L-Histidine monohydrochloride monohydrate; 0.053% (w/v) Polysorbate 80 and the TNF-α inhibitor used herein may be an anti-TNF-α antibody formulated in an aqueous solution at 100 mg/mL: 4.1% (w/v) sorbitol, 5.6 mM L-Histidine and L-Histidine monohydrochloride monohydrate; 0.015% (w/v) Polysorbate 80.

Prior to administration, a combination of the anti-IL-23p19 antibody and the anti-TNF-α antibody at a desired ratio may be obtained by mixing appropriate amount of the two antibody solutions. For example, a combination of the anti-IL-23p19 antibody and the anti-TNF-α antibody at 2:1 (w/w) ratio may be obtained by mixing 2 mL of the solution containing 100 mg/mL anti-IL-23p19 antibody and 1 mL of the solution containing 100 mg/mL anti-TNF-α antibody. The resulting 3 mL mix contains 200 mg of the anti-IL-23p19 antibody and 100 mg of the anti-TNF-α antibody. For patients receiving a combination of 160 mg of the anti-IL-23p19 antibody and 80 mg of the anti-TNF-α antibody will be subcutaneously administered 2.4 mL of the mix. In another example, a combination of the anti-IL-23p19 antibody and the anti-TNF-α antibody at 1:1 (w/w) ratio may be obtained by mixing 1 mL of the solution containing 100 mg/mL anti-IL-23p19 antibody and 1 mL of the solution containing 100 mg/mL anti-TNF-α antibody. The resulting 2 mL mix contains 100 mg of the anti-IL-23p19 antibody and 100 mg of the anti-TNF-α antibody. For patients receiving a combination of 160 mg of the anti-IL-23p19 antibody and 80 mg of the anti-TNF-α antibody will be subcutaneously administered 1.6 mL of the mix and 0.8 mL of the solution containing 100 mg/mL anti-IL-23p19 antibody.

In another aspect is provided a kit comprising a combination of an IL-23 inhibitor (e.g., an anti-IL-23p19 antibody) and a TNF-α inhibitor (e.g., an anti-TNF-α antibody) and instructions for using the same to treat CD in a subject (e.g., a human patient suffering from moderately or severely active CD). The instruction may contain guidance on handling the medication and dosing regimen. For example, the instruction may contain guidance on subcutaneously administering to the subject (i) about 320 mg of the IL-23 inhibitor and about 160 mg of the TNF-α inhibitor at weeks 0, 4, and 8; (ii) about 160 mg of the IL-23 inhibitor and about 80 mg of the TNF-α inhibitor at weeks 0, 4, and 8; (iii) about 160 mg of the IL-23 inhibitor and about 80 mg of the TNF-α inhibitor every 4 weeks; (iv) about 40 mg of the IL-23 inhibitor and about 40 mg of the TNF-α inhibitor every 4 weeks; or (v) about 20 mg of the IL-23 inhibitor and about 20 mg of the TNF-α inhibitor every 4 weeks.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Clinical Study of Combination Therapy with Guselkumab and Golimumab in Participants with Moderately to Severely Active Crohn's Disease

This is a phase 2b randomized, double-blind, active- and placebo-controlled, parallel-group, multicenter study to evaluate the efficacy and safety of induction and maintenance combination therapy with guselkumab and golimumab in participants with moderately to severely active CD.

Guselkumab (TREMFYA®) is a fully human immunoglobulin G (IgG) 1 lambda monoclonal antibody (mAb) that binds to the p19 subunit of human interleukin (IL)-23 with high specificity and affinity. The binding of guselkumab to IL-23 blocks the binding of extracellular IL-23 to the cell surface IL-23 receptor, inhibiting IL-23-specific intracellular signaling and subsequent activation and cytokine production. In this manner, guselkumab inhibits the biological activity of IL-23 in all in vitro assays examined.

Guselkumab is currently approved for the treatment of adults with moderate-to-severe plaque psoriasis or active psoriatic arthritis (PsA), in the United States (US), European Union (EU), Canada, and several countries in Latin America, and the Asia-Pacific region. Guselkumab has also been approved for the treatment of generalized pustular psoriasis, erythrodermic psoriasis, and palmoplantar pustulosis in Japan.

Golimumab (SIMPONI®) is a fully human anti-tumor necrosis factor alpha (TNF-α) mAb that binds to TNF-α with high affinity. This interaction prevents the binding of TNF-α to its receptors, thereby inhibiting the biological activity of TNF-α. The overall anti-TNFα activity results in limited production or activity of inflammatory cytokines, thereby providing therapeutic benefit in various chronic inflammatory disorders, including UC.

Golimumab administered subcutaneously (SC), is approved for the treatment of moderately to severely active UC in over 100 countries worldwide. Additionally, golimumab (either SIMPONI® or SIMPONI ARIA® [administered IV]) is approved for 1 or more of the following indications around the world: rheumatoid arthritis, PsA, ankylosing spondylitis, nonradiographic axial spondyloarthritis, and polyarticular juvenile idiopathic arthritis.

In the present clinical study, a combination or coformulation of guselkumab and golimumab (guselkumab/golimumab coformulation) is administered in patients with moderately to severely CD. The coformulation of guselkumab and golimumab allows both interventions to be delivered in a single administration.

OBJECTIVES AND ENDPOINTS

| | |
|---|---|
| Objectives | Endpoints |

Primary

| | |
|---|---|
| To evaluate the efficacy of the | Clinical remission at Week 48 |
| combination of guselkumab and | Endoscopic response at Week 48 |
| golimumab at Week 48 compared | |
| with each monotherapy (guselkumab | |
| alone and golimumab alone) | |

Secondary

| | |
|---|---|
| To evaluate the efficacy of the | Patient-reported Outcomes (PRO)-2 |
| combination of guselkumab and | remission at Week 48 |
| golimumab compared with each | Endoscopic remission at Week 48 |
| monotherapy across a range of outcomes | |
| To evaluate the efficacy of the | Clinical remission at Week 24 |
| combination of guselkumab and | Endoscopic response at Week 24 |
| golimumab at Week 24 compared with | |
| placebo | |
| To evaluate the safety based on AEs | Frequency and type of adverse events |
| of the combination of guselkumab | (AEs) and serious adverse events (SAEs) |
| and golimumab compared with each | |
| monotherapy and placebo | |
| To evaluate the pharmacokinetics (PK) | Serum concentrations of guselkumab |
| and immunogenicity of the combination | over time |
| of guselkumab and golimumab compared | Serum concentrations of golimumab |
| with each monotherapy | over time |
| | Incidence and titers of antibodies to |
| | guselkumab |
| | Incidence and titers of antibodies to |
| | golimumab. |
| | Incidence of neutralizing antibodies to |
| | guselkumab |
| | Incidence of neutralizing antibodies to |
| | golimumab |

Exploratory

| | |
|---|---|
| To evaluate the dose-response of | Based on the totality of data, examining |
| guselkumab/golimumab coformulation | efficacy, safety, PK, and exposure- |
| to inform dose selection for the | response |
| Phase 3 study. | |
| To evaluate the impact of the | Laboratory parameters and change from |
| combination of guselkumab and | baseline in laboratory parameters |
| golimumab compared with each | (hematology and chemistry) |
| monotherapy on other safety | Vital signs |
| endpoints | Columbia-Suicide Severity Rating Scale |
| | (C-SSRS) |
| To evaluate the impact of the | Endpoints based on clinical remission at |
| combination of guselkumab and | multiple time points |
| golimumab compared with each | Endpoints based on endoscopic |
| monotherapy on sustained efficacy | definitions at multiple time points |
| To evaluate the impact of the | Endpoints based on CDAI, PRO-2, SF, |
| combination of guselkumab and | AP, and Crohn's Disease Patient-reported |
| golimumab compared with each | Outcome Signs and Symptoms (CD- |
| monotherapy on symptoms and the | PRO/SS), including corticosteroid free |
| use of corticosteroids | remission over a range of time points |
| To evaluate the impact of the | Endpoints based on C-reactive protein |
| combination of guselkumab and | (CRP) and fecal calprotectin |
| golimumab compared with each | measurements |
| monotherapy on inflammatory | |
| biomarkers | |
| To evaluate the impact of combination | Endpoints based on Inflammatory Bowel |
| of guselkumab and golimumab compared | Disease Questionnaire (IBDQ), Patient- |
| with each monotherapy on health- | reported Outcomes Measurement |
| related quality of life | Information System (PROMIS-29), and |
| | Work Productivity and Activity |
| | Impairment in Crohn's Disease |
| | (WPAI-CD) |
| To evaluate the impact of the | Endoscopic remission, based on |
| combination of guselkumab and | alternative definition |
| golimumab compared with each | Endpoints based on histologic |
| monotherapy on endoscopic and | assessments |
| histologic improvement | Endpoints based on the SES-CD and |
| | absence of ulceration |
| | Assessment of conventional GI |
| | evaluations (ileocolonoscopy video |
| | imaging and mucosal histology images |
| | assessed by trained or certified providers) |
| | and comparison to novel artificial |
| | intelligence technologies |

### Efficacy Endpoints Definitions are as Follows:

Clinical remission: CDAI score <150
Endoscopic response: A ≥50% improvement from baseline in Simple Endoscopic Score for Crohn's Disease [SES-CD] score
Patient-reported Outcomes (PRO)-2 remission: an AP mean daily score ≤1 and stool frequency (SF) mean daily score ≤3, and no worsening of AP or SF from baseline
Endoscopic remission:
The endoscopic remission is defined as; SES-CD score ≤4 with at least 2 points reduction from baseline and no subscore >1 in any individual component.
The alternative definition of endoscopic remission is defined as: SES-CD score ≤2.

The Crohn's Disease Activity Index (CDAI) will be assessed by collecting information on 8 different CD-related variables: extraintestinal manifestations, abdominal mass, weight, hematocrit, total number of liquid or very soft stools, AP/cramping, use of antidiarrheal drug(s) and/or opiates, and general well-being. Total number of liquid or very soft stools, AP/cramping and general well-being are recorded by the participant on an electronic home diary completed on a daily basis leading up to a visit. Participants will complete the home diary daily for 10 days leading up to the Week 0 visit and through the Week 8 visit. Subsequently, participants will complete the home diary for 10 days prior to each visit. The PRO-2 includes the CDAI components of the total number of liquid or very soft stools and the AP score.

### Overall Design

This is a randomized, double-blind, placebo- and active-controlled, parallel-group, multicenter study to evaluate the efficacy and safety of induction and maintenance combination therapy with guselkumab and golimumab in participants with moderately to severely active CD who have had an inadequate initial clinical response, loss of clinical response, or intolerance to ≥1 previous approved advanced therapy ADT (advanced therapy inadequate responder [ADT-IR]). ADT is defined as a biologic (including anti-TNFα, ustekinumab, or vedolizumab as branded or as a biosimilar). ADT is used to distinguish these agents from conventional therapy, which consists of corticosteroids and immunomodulators. This dose-ranging study targets participants 18 to 65 years of age (inclusive, at the time of consent) with moderately or severely active CD (defined by a CDAI score ≥220 and ≤450) and either a mean daily AP score ≥2 (based on the unweighted CDAI component of AP) or a mean daily SF count ≥4 (based on the unweighted CDAI component of the number of liquid or very soft stools), of at least 3 months duration, with colitis, ileitis, or ileocolitis previously confirmed in the past by radiology, histology and/or endoscopy. Participants must also have endoscopic evidence of active ileal and/or colonic CD (screening SES-CD score>6 [or >4 for isolated ileal disease]) at the time of screening ileocolonoscopy.

A target of 715 participants will be enrolled in this study with 65 participants planned in the placebo group and 130 participants planned per active intervention group. Participants will be screened for study eligibility within 6 weeks prior to randomization at the Week 0 visit. Eligible participants will then be randomized in a 1:2:2:2:2:2 ratio to one of the following:
Group 1 (Placebo group): placebo SC at Weeks 0, 4, and 8 followed by placebo SC every 4 weeks (q4w)
Group 2 (Guselkumab monotherapy group): guselkumab 400 mg SC at Weeks 0, 4, and 8 followed by guselkumab 200 mg SC q4w
Group 3 (Golimumab monotherapy group): golimumab 200 mg SC at Weeks 0 and 4 followed by golimumab 100 mg SC q4w
Group 4 (High-Dose Combination Regimen): guselkumab 320 mg and golimumab 160 mg SC at Weeks 0, 4, and 8 followed by the combination of guselkumab 160 mg and golimumab 80 mg SC q4w
Group 5 (Middle-Dose Combination Regimen): guselkumab 320 mg and golimumab 160 mg SC at Weeks 0, 4, and 8 followed by the combination of guselkumab 40 mg and golimumab 40 mg SC q4w
Group 6 (Low-Dose Combination Regimen): guselkumab 160 mg and golimumab 80 mg SC at Weeks 0, 4, and 8 followed by the combination of guselkumab 20 mg and golimumab 20 mg SC q4w

Eligible participants will be allocated to a treatment group using permutated block randomization stratified by baseline CDAI (≤300, >300), SES-CD score (≤12, >12), and history of primary nonresponse to at least one approved ADT (Yes, No). Visits will occur in person q4w for safety and efficacy assessments, as well as administration of study intervention. Two telephone visits will also occur at Weeks 2 and 6 for additional safety and symptom assessments. Participants who are inadequate responders at Week 24 (which is defined as meeting all 3 conditions: [1] at Week 20, a CDAI score ≥220 and <70 point reduction from baseline CDAI; [2] at Week 24, a CDAI score ≥220 and <70 point reduction from baseline; and [3] a reduction from baseline of SES-CD<25% at Week 24) will receive the following treatment escalation based on their initial study intervention group assignment:
Group 1 (placebo group), Group 2 (Guselkumab monotherapy group), Group 3 (Golimumab monotherapy group), Group 6 (Combination low-dose group), will receive the combination of guselkumab mid-dose induction and maintenance: guselkumab 320 mg and golimumab 60 mg SC co-formulation at Weeks 24, 28, and 32 followed by guselkumab 40 mg and golimumab 40 mg SC co-formulation q4w
Group 4 (Combination high-dose group) and Group 5 (Combination mid-dose group) will receive the combination of guselkumab and golimumab high-dose induction and maintenance: guselkumab 320 mg and golimumab 160 mg SC co-formulation at Weeks 24, 28, and 32 followed by guselkumab 160 mg and golimumab 80 mg SC co-formulation q4w

Baseline is defined as Week 0.

One interim analysis (IA) is planned in the study. The IA will be a futility analysis to be conducted after the first 250 randomized and treated participants (approximately 35%) have reached Week 12 (or have terminated study participation prior to the Week 12 visit) and at least the first 110 randomized and treated participants have reached Week 24 (or have terminated study participation before the Week 24 visit).

Two database locks (DBLs) are planned for the main study (ie, through Week 48). The first DBL is for the IA. The second DBL is the Primary Analysis DBL and is planned for Week 48 when all randomized participants reach Week 48 (or have terminated study participation before the Week 48 visit).

Permitted concomitant medications include: (1) oral corticosteroids at a prednisone-equivalent dose of ≤20 mg/day if participants have been on a stable dose for ≥2 weeks; and (2) conventional immunomodulators (ie, azathioprine [AZA], 6-mercaptopurine [6-MP], or methotrexate [MTX]) if participants have been taking them for ≥12 weeks and have been on a stable dose for ≥4 weeks. All participants who were taking corticosteroids at Week 0 may begin tapering them as early as Week 8 but must initiate tapering no later than Week 12 (provided it is medically feasible). Corticosteroids and immunomodulators should not be initiated or increased above baseline doses.

Participants will complete an early discontinuation (ED) visit upon discontinuation of study intervention and before termination of study participation. All randomized and treated participants are to complete the safety follow-up visit 12 weeks after the last dose of study intervention.

Efficacy, safety, PK, immunogenicity, PROs, and biomarkers (where local regulations permit) will be assessed according to the Schedule of Activities.

Safety assessments include AEs, clinical laboratory tests (hematology and chemistry), vital signs, physical examination, screening electrocardiogram (ECG), suicidality assessment, and monitoring for hypersensitivity reactions, injection-site reactions, and early detection of active TB.

An external independent data monitoring committee (DMC) will be commissioned for this study to monitor participant safety and evaluate the futility analysis.

### High-Dose Combination Regimen

Several factors were considered for the selection of this high-dose combination regimen. First, it aims to maximize the efficacy for the combination therapy by using doses that are close to the monotherapy doses that are anticipated to provide optimal benefit-risk and, in the case of guselkumab, close to the higher monotherapy doses that are being assessed in Phase 3 studies. The doses of guselkumab and golimumab in the high-dose combination regimen represent a 20% reduction from the respective monotherapy doses. The reduction in dose amount (20% lower) is mainly to help co-formulate the 2 mAbs so that a smaller volume can be delivered on a synchronized schedule. Despite the use of slightly lower doses in the combination, the combination therapy is expected to have superior efficacy over the respective monotherapies due to the dual inhibition of both IL-23 and TNF-α. In addition, dosing frequency of both mAbs is harmonized to make the co-formulation feasible.

Compared with the guselkumab 200 mg IV induction dose and the guselkumab 400 mg SC induction dose being tested in the Phase 3 as monotherapy, the proposed guselkumab 320 mg SC induction for the high-dose combination regimen would result in a slightly higher trough concentration, despite a 20% lower AUC in the 12-week induction period. In the case of the guselkumab mono-component in the top maintenance regimen, the proposed dose (160 mg SC q4w) would provide a PK exposure approximately 20% lower than that of the higher of the 2 maintenance doses (200 mg SC q4w) that are currently being studied in the Phase 3 monotherapy study.

Golimumab 160 mg SC given at Weeks 0, 4, and 8 has been selected as the top induction combination dose because it is expected to provide a similar cumulative dose amount (480 mg vs 500 mg), similar cumulative AUC, peak, and trough concentration in the 12-week induction period compared with the approved posology of golimumab as monotherapy in UC (200 mg SC at Week 0, 100 mg SC at Week 2, and 100 mg SC q4w thereafter). In terms of the golimumab maintenance dose, both 50 mg q4w and 100 mg q4w were demonstrated to be safe and efficacious as UC monotherapy in PURSUIT-M; 100 mg q4w showed a small incremental benefit in participants weighing ≥80 kg for long-term clinical remission (Sandborn et al., Subcutaneous golimumab maintains clinical response in patients with moderate-to-severe ulcerative colitis, Gastroenterology. 2014 January; 146 (1): 96-109.el. doi: 10.1053/j.gastro.2013.06.010. Epub 2013 June). PK simulations suggest that an intermediate combination-specific dose of golimumab 80 mg q4w would achieve overlapping drug exposure versus the approved maintenance dose for the treatment of UC regardless of participants' body weight.

### Middle-Dose Combination Regimen

The proposed middle-dose combination regimen consists of the same induction doses of guselkumab and golimumab used in the high-dose combination regimen (320 mg/160 mg) but a lower maintenance dose of guselkumab 40 mg and golimumab 40 mg SC q4w. matching the high and middle maintenance combination doses to the same induction combination dose would allow for a better characterization of the maintenance dose-response.

The maintenance dose of guselkumab mono-component of 40 mg SC q4w is selected to approximate the exposures observed with the lower of the 2 maintenance doses being studied in the Phase 3 monotherapy study (ie, 100 mg q8w). Guselkumab 40 mg q4w dose is predicted to produce steady-state trough levels slightly higher than those with 100 mg q8w despite a 20% lower AUC per week. Trough concentration is an important predictor of maintenance efficacy in IBD (Sheasgreen et al., The Evolving Evidence for Therapeutic Drug Monitoring of Monoclonal Antibodies in Inflammatory Bowel Disease, Curr Gastroenterol Rep. 2017 May; 19 (5): 19. doi: 10.1007/s11894-017-0559-8; Hoseyni et al., Therapeutic Drug Monitoring of Biologics for Inflammatory Bowel Disease: An Answer to Optimized Treatment? J Clin Pharmacol. 2018 July; 58(7): 864-876. doi: 10.1002/jcph.1084. Epub 2018 Feb. 20; Argollo et al., Optimizing biologic therapy in IBD: how essential is therapeutic drug monitoring? Nat Rev Gastroenterol Hepatol. 2020 November; 17 (11): 702-710). As a result, it is expected that guselkumab 40 mg q4w may produce efficacy comparable with that of guselkumab 100 mg q8w.

The golimumab maintenance dose used in the middle-dose combination regimen is 40 mg q4w, 20% lower than the 50 mg q4w dose which was efficacious as monotherapy in UC (PURSUIT-M in Sandborn et al. 2014). It is included to test whether the golimumab dose lower than the monotherapy can produce the desired efficacy when used in combination.

### Low-Dose Combination Regimen

In the low-dose combination regimen, both induction and maintenance doses for guselkumab and golimumab are reduced by 50% compared with the middle-dose combination regimen. This low-dose combination regimen aims to explore whether combination doses, which are substantially lower than the respective monotherapy doses, can achieve the desired efficacy with the potential to enhance the overall benefit/risk ratio.

### Study Intervention

All participants will receive SC injections q4w via an infusion set. In order to maintain the blind, all study intervention will be prepared into the same number of syringes with matching volume. During induction (Weeks 0, 4, and 8), 4 syringes will be prepared while 3 syringes of each study intervention will be prepared during maintenance. Study intervention preparation will be performed by an unblinded pharmacist. Study intervention will then be administered SC via an infusion set in order to limit the number of needlesticks. There will be two needlesticks during induction and one needlestick during maintenance.

Study intervention can only be administered at the sites.

Guselkumab/golimumab coformulation, guselkumab, and golimumab will be manufactured and provided under the responsibility of the sponsor. Phase 2b doses will use a “Mix and Delivery” approach by which a guselkumab/golimumab coformulation prefilled syringe (PFS) product containing 100 mg guselkumab and 100 mg golimumab, plus individual guselkumab 100 mg PFS and/or ImL placebo PFS will be used to provide the desired dose levels for the combination groups. This ‘mix and delivery’ system will enable blinding as the number and volume of injections will be the same for all groups. Separate guselkumab 100 mg PFS or golimumab 100 mg PFS product will be used to provide the desired dose levels for the monotherapy groups with proper mix of placebo PFS(s). The placebo group will receive placebo in the same number of syringes and volumes that will match the active treatment groups.

The study interventions will be supplied to the pharmacist/qualified site designee as follows:
Guselkumab will be supplied as a 100 mg/mL sterile liquid in a single-dose PFS assembled in an UltraSafe Plus.
Golimumab will be supplied as a 100 mg/mL sterile liquid in a single-use PFS assembled in an UltraSafe.
Guselkumab/golimumab coformulation will be supplied as a 2 mL sterile liquid containing 100 mg of guselkumab and 100 mg of golimumab in a single-dose PFS assembled in an UltraSafe Plus, which contains the same excipients as the individual monotherapy components at lower concentrations.
The 1 mL placebo PFS will be supplied as a 1 mL sterile liquid in a single-use PFS assembled in an UltraSafe Plus, which has the same excipients as the guselkumab 100 mg PFS but without active intervention.
The 2 mL placebo PFS will be supplied as a 2 mL sterile liquid in a single-use PFS assembled in an UltraSafe Plus, which has the same excipients as the guselkumab 100 mg PFS but without active intervention
A 20 mL placebo vial will be supplied which has the same excipients as the guselkumab 200 mg final vialed product without active intervention.

### Study Assessments

### Efficacy Assessments

Efficacy assessments will include the following:
CDAI
PRO-2 (the stool frequency and abdominal pain elements of the CDAI)
Endoscopic assessments of the intestinal mucosa based on the presence and absence of mucosal ulcerations and the SES-CD, and histologic assessments based on the Global Histology Activity Score (GHAS), Geboes score, and Robarts Histopathology Index
Fistula assessment
Inflammatory PD markers, including CRP and fecal calprotectin
PRO measures to assess HRQOL and work productivity outcomes including IBDQ, PROMIS-29, CD-PRO/SS, and WPAI-CD
extraintestinal manifestations (EIMs)

PRO instruments will be provided in the local language in accordance with local guidelines. The PRO and AE data will not be reconciled with one another.

### Safety Assessments

Safety evaluations will include the assessment of AEs, clinical laboratory tests (hematology and chemistry), vital signs, physical examinations, screening ECG, concomitant medication review, and monitoring for hypersensitivity reactions, injection-site reactions, suicidal ideation and behavior by the C-SSRS, and early detection of active TB.

Adverse events will be reported and followed by the investigator.

Any clinically relevant changes occurring during the study must be recorded on the Adverse Event section of the Case Report Form (CRF).

Any clinically significant abnormalities persisting at the end of the study/early withdrawal will be followed by the investigator until resolution or until a clinically stable condition is reached.

### Pharmacokinetics Assessments

Serum samples will be used to evaluate the PK of guselkumab and golimumab. Serum collected for PK may additionally be used to evaluate safety or efficacy aspects that address concerns arising during or after the study period. Genetic analyses will not be performed on these serum samples. Participant confidentiality will be maintained.

### Genetics

A pharmacogenomic blood sample will be collected from participants who consent separately to this component of the study to allow for pharmacogenomic research, as necessary. Participant participation in pharmacogenomic research is optional.

Genetic (DNA) variation may be an important contributory factor to interindividual variability in drug response and associated clinical outcomes. Genetic factors may also serve as markers for disease susceptibility and prognosis and may identify population subgroups that respond differently to an intervention. DNA samples will be analyzed for identification of genetic factors that may be associated with clinical response. This research may consist of the analysis of 1 or more candidate genes, assessment of single nucleic polymorphisms (SNPs) in relation to guselkumab and/or guselkumab intervention and/or Crohn's disease. Whole genome sequencing will not be performed. Whole blood samples will be collected for genetic analyses.

### Biomarkers

Biomarker assessments will be made to examine the biologic response to treatment and to identify biomarkers that are relevant to guselkumab and/or golimumab in the treatment of CD. Combination TNF-α and IL-23 blockade will be compared to the selective inhibition of TNF-α or IL-23. Assessments will include the evaluation of relevant biomarkers in serum, whole blood, stool, and mucosal biopsy samples collected as scheduled. Data collected from these samples will be used for exploratory research that will include the following objectives:
1. To understand the molecular effects of guselkumab and golimumab combination treatment or monotherapy of either agent.
2. To understand CD pathogenesis.
3. To understand why an individual participant may respond differently to guselkumab or golimumab.
4. To understand the impact of treatment with guselkumab and golimumab treatment, alone and in combination on intestinal inflammation.
5. To develop diagnostic tests to identify CD populations that may be responsive or non-responsive to treatment with guselkumab/golimumab combination therapy, guselkumab, and golimumab.

Biomarker analyses are dependent upon the availability of appropriate biomarker assays and clinical response rates. Biomarker analysis may be deferred or not performed, if during or at the end of the study, it becomes clear that the analysis will not have sufficient scientific value for biomarker evaluation, or if there are not enough samples or responders to allow for adequate biomarker evaluation. In the event the study is terminated early or shows poor clinical efficacy, completion of biomarker assessments is based on justification and intended utility of the data.

### Immunogenicity Assessments

Antibodies to guselkumab and/or golimumab will be evaluated in serum samples collected from all participants. Additionally, serum samples should also be collected at the final visit from participants who discontinued study intervention or were withdrawn from the study. These samples will be tested by the sponsor or sponsor's designee.

Serum samples will be screened for antibodies binding to guselkumab and/or golimumab and the titer of confirmed positive samples will be reported. Other analyses may be performed to verify the stability of antibodies to guselkumab and/or golimumab and/or further characterize the immunogenicity of guselkumab and/or golimumab.

Serum samples will be used to evaluate the immunogenicity of anti-guselkumab and/or anti-golimumab antibodies. Samples collected for immunogenicity analyses may additionally be used to evaluate safety or efficacy aspects that address concerns arising during or after the study period. Genetic analyses will not be performed on these serum samples. Participant confidentiality will be maintained.

The detection and characterization of antibodies to guselkumab and/or golimumab will be performed using a validated assay method by or under the supervision of the sponsor. All samples collected for detection of antibodies to guselkumab and/or golimumab will also be evaluated for guselkumab and/or golimumab serum concentration to enable interpretation of the antibody data. Antibodies may be further characterized and/or evaluated for their ability to neutralize the activity of the study interventions. Samples may be stored up to 15 years (or according to local regulations) following the last participant's last visit for the study at a facility selected by the sponsor to enable further analysis of immune responses to guselkumab and/or golimumab.

### Medical Resource Utilization and Health Economics

Medical resource utilization and health economics data, associated with medical encounters, will be collected in the CRF by the investigator and study site personnel for all participants throughout the study. Protocol-mandated procedures, tests, and encounters are excluded. The data collected may be used to conduct exploratory economic analyses and will include:
Number and duration of medical care encounters, including surgeries, and other selected procedures (inpatient and outpatient).
Duration of hospitalization (total days length of stay, including duration by wards; (eg, intensive care unit)
Number and character of diagnostic and therapeutic tests and procedures
Outpatient medical encounters and treatments (including physician or emergency room visits, tests and procedures, and medications).

### Statistical Methods

Statistical analysis will be done by the sponsor or under the authority of the sponsor. A general description of the statistical methods to be used to analyze the efficacy and safety data is outlined below.

### Sample Size Determination

Sample size was determined to provide sufficient power to detect treatment differences in the co-primary endpoint, clinical remission at Week 48 and endoscopic response at Week 48, between the combination of guselkumab and golimumab relative to both guselkumab alone and golimumab alone.

Taking into account the intercurrent events and analysis strategies, the clinical remission rates at Week 48 are assumed to be 70% for the combination high-dose group, 50% for guselkumab, and 30% for golimumab, and endoscopic response rates at Week 48 are assumed to be 55% for the combination high-dose group, 35% for guselkumab and 18% for golimumab. Based on these assumptions with 130 participants per active treatment group, there is >80% power to detect treatment differences in both primary endpoints when the combination high-dose group is compared with each of the monotherapies at alpha=0.05 (2-sided). A multiplicity-controlled testing procedure will be implemented for the co-primary and secondary endpoints, regarding the different combination regimens.

The assumptions for the sample size calculations were based on data from a Phase 2 study, in which the efficacy and safety of guselkumab in CD were assessed in a 48-week treat-through study in patients with moderate to severely active CD. Since golimumab has not been previously studied in CD, those assumptions were built on previous results from adalimumab studies, as an anti-TNFα that has been studied in CD in participants with inadequate response to infliximab (Colombel et al., Adalimumab for maintenance of clinical response and remission in patients with Crohn's disease: the CHARM trial. Gastroenterology. 2007; 132(1): 52-65; Sandborn et al., Adalimumab induction therapy for Crohn disease previously treated with infliximab: a randomized trial. Ann Intern Med. 2007; 146(12): 829-838; and Panaccione et al. Clinical Benefit of Long-Term Adalimumab Treatment in Patients With Crohn's Disease Following Loss of Response or Intolerance to Infliximab: 96-Week Efficacy Data From GAIN/ADHERE Trials. J Crohns Colitis. 2018; 12(8): 930-938) and a 52-week treat-through study that compared ustekinumab to adalimumab in patients with moderate to severely active CD (Irving et al. OP02 Ustekinumab versus adalimumab for induction and maintenance therapy in Moderate-to-Severe Crohn's Disease: The SEAVUE study J Crohns Colitis 2021; 15: S001-002). The rates of combination therapy were estimated as compared to the 2 individual monotherapies.

Table 3 provides the power evaluations for the co-primary endpoints under various scenarios for a total sample size of 715 participants (65 participants in placebo group and 130 per active treatment group). The bolded assumptions are considered the base case.

Table 4 and Table 5 provide the power evaluations for each co-primary endpoint: clinical remission at Week 48 and endoscopic response at Week 48, respectively, for a total sample size of 715 participants (65 participants in placebo group and 130 per active treatment group).

TABLE 3
Power Evaluation for the Co-primary endpoints with N = 130 per active treatment group at alpha = 0.05 (2-sided)

| | | | | | | | | Combo high- |
|---|---|---|---|---|---|---|---|---|
| Clinical | Endoscopic | Clinical | Endoscopic | Clinical | Endoscopic | Combo High- | Combo High- | dose vs |
| remission | response | remission | response | remission | response | dose vs | dose vs | Golimumab AND |
| at Wk 48 | at Wk 48 | at Wk 48 | at Wk 48 | at Wk 48 | at Wk 48 | Golimumab | Guselkumab | Guselkumab |
| 30% | 18% | 50% | 35% | 70% | 55% | >99% | 83% | 83% |
| 30% | 18% | 50% | 35% | 65% | 55% | >99% | 63% | 63% |
| 30% | 18% | 50% | 35% | 70% | 50% | >99% | 63% | 63% |
| 30% | 18% | 50% | 35% | 65% | 50% | >99% | 47% | 47% |
| 30% | 18% | 50% | 35% | 60% | 45% | >99% | 14% | 14% |

Note:
The power table was based on simulations, where clinical remission at Week 48 and endoscopic response at Week 48 were assumed to be independent due to their weak correlation. Wald test was used for treatment comparisons in the simulations.

TABLE 4
Power Evaluation for the primary endpoint: clinical remission at Week 48 with
N = 130 per active treatment group at alpha = 0.05 (2-sided)

| Clinical remission at Wk 48 | | | Power | | |
|---|---|---|---|---|---|
| Golimumab | Guselkumab | Combo | Combo | Combo | Combo high-dose |
| (monotherapy | (monotherapy | high- | high-dose vs | high-dose vs | vs Golimumab |
| alone) | alone) | dose | Golimumab | Guselkumab | AND Guselkumab |
| 30% | 50% | 70% | >99% | 91% | 91% |
| 30% | 50% | 68% | >99% | 84% | 84% |
| 30% | 50% | 65% | >99% | 69% | 69% |
| 30% | 50% | 60% | >99% | 38% | 38% |

Note:
The power table was based on simulations. The Wald test was used for treatment comparisons in the simulations.

TABLE 5
Power Evaluation for the primary endpoint: endoscopic response at Week 48 with
N = 130 per active treatment group at alpha = 0.05 (2-sided)

| Endoscopic response at Wk 48 | | | Power | | |
|---|---|---|---|---|---|
| Golimumab | Guselkumab | | Combo | Combo | Combo high-dose |
| (monotherapy | (monotherapy | Combo | high-dose vs | high-dose vs | vs Golimumab |
| alone) | alone) | high-dose | Golimumab | Guselkumab | AND Guselkumab |
| 18% | 35% | 55% | >99% | 91% | 91% |
| 18% | 35% | 53% | >99% | 84% | 84% |
| 18% | 35% | 50% | >99% | 70% | 70% |
| 18% | 35% | 45% | >99% | 38% | 38% |

Note:
The power table was based on simulations. The Wald test was used for treatment comparisons in the simulations.

### Statistical Analyses

Descriptive statistics (eg, mean, median, standard deviation [SD], interquartile [IQ] range, minimum, and maximum) will be used to summarize continuous variables. Counts and percentages will be used to summarize categorical variables. Graphical data displays (eg, line plots) may also be used to summarize data.

Analyses suitable for categorical data (eg, chi-square tests, Cochran-Mantel-Haenszel [CMH] chisquare tests, or logistic regression, as appropriate) will be used to compare the proportions of participants achieving selected endpoints (eg, clinical response). In cases of rare events, the Fisher's exact test will be used for treatment comparisons. Continuous response parameters will be compared using an analysis of variance (ANOVA) or analysis of covariance (ANCOVA), unless otherwise specified. If the normality assumption is in question, an ANOVA or ANCOVA on the van der Waerden normal scores will be used.

The overall Type I error rate will be controlled at the significance level of 0.05 (2-sided).

### Primary Endpoint

The co-primary endpoints are clinical remission (CDAI score <150) at Week 48 and endoscopic response (≥50% improvement from baseline in the SES-CD score) at Week 48. The analyses will be based on the Full Analysis Set, defined as all randomized participants who had at least one study intervention administration. Participants will be analyzed according to the study intervention group to which they were randomized regardless of the study intervention they received.

The primary estimands, ie, a precise definition of the primary targeted treatment effect, are defined by 5 attributes (treatment, population, variable, intercurrent events [ICE], and population-level summary) for the primary endpoint as stated below.
Primary Estimand of Clinical Remission at Week 48

### Treatment by Week 48:

### Experimental:

Combination high-dose group: guselkumab 320 mg and golimumab 160 mg SC at Weeks 0, 4, and 8 followed by the combination of guselkumab 160 mg and golimumab 80 mg SC q4w
Combination mid-dose group: guselkumab 320 mg and golimumab 160 mg SC at Weeks 0, 4, and 8 followed by the combination of guselkumab 40 mg and golimumab 40 mg SC q4w
Combination low-dose group: guselkumab 160 mg and golimumab 80 mg SC at Weeks 0, 4, and 8 followed by the combination of guselkumab 20 mg and golimumab 20 mg SC q4w

### Control:

Guselkumab monotherapy group: guselkumab 400 mg SC with dosing at Weeks 0, 4, and 8 followed by guselkumab 200 mg SC q4w
Golimumab monotherapy group: golimumab 200 mg SC with dosing at Weeks 0, and 4 followed by golimumab 100 mg SC q4w

The placebo group is not included in this estimand.

Population: Participants with moderately to severely active CD who have had an inadequate initial clinical response, loss of clinical response, or intolerance to ≥1 previous ADT.

Variable (endpoint): Binary response variable (response/non-response) with response defined as achieving a CDAI score <150 at Week 48 without experiencing any of the ICEs in categories 1 to 5 as outlined below prior to Week 48 visit.

Intercurrent Events: Table 6 describes the ICEs and corresponding analysis strategies.

TABLE 6
Intercurrent Events and Corresponding Analysis
Strategies for Clinical Remission at Week 48

| Intercurrent Events | Analysis Strategy |
|---|---|
| (between baseline and Week 48) | for Intercurrent Events |
| 1. A CD-related surgery (with the exception | Composite Strategy: Participants are considered |
| of minor procedures such as drainage of a | non-responders if they experience any of these |
| superficial abscess or seton placement, etc.) | ICEs, prior to Week 48, as reflected in the |
| | variable definition. |
| 2. Prohibited changes in CD medication(s). | |
| 3. Escalated treatment due to meeting the | |
| criteria of inadequate response at Week 24. | |
| 4. Discontinuation of study intervention | |
| due to lack of efficacy, AE of disease | |
| worsening. | |
| 5. Discontinuation of study intervention | |
| due to COVID-19 infection or for reasons | |
| other than those included in ICE 4 and ICE 6. | |
| 6. Discontinuation of study intervention | Hypothetical Strategy: Data points after |
| due to COVID-19 related reasons (excluding | the occurrence of ICE6 and onward will |
| COVID-19 infection). | not be used. |

Abbreviations:
AE = adverse event;
COVID-19 = Coronavirus disease 2019;
ICE = intercurrent event

Population-level Summary: differences in the proportion of participants who achieved the binary response at Week 48 as defined in the Variable attribute above between each of the combination groups and each of the monotherapy groups.

### Primary Estimand of Endoscopic Response at Week 48

The attributes are the same as those for the primary estimand of clinical remission at Week 48 with the exception of the Variable (Endpoint), which are described as follows:

Variable (endpoint): Binary response variable (response/non-response) with response defined as achieved a ≥50% improvement from baseline in the SES-CD score at Week 48 without experiencing any of the ICEs in categories 1 to 5 (as outlined in the ICE attribute in the primary estimand of clinical remission at Week 48) prior to the Week 48 visit.

### Estimator for the Co-Primary Endpoints

Summaries of the proportion of participants in clinical remission by treatment group, the adjusted treatment difference (with CMH stratified by randomization stratification factors) between each combination therapy group versus each monotherapy group, as well as the associated 95% confidence interval will be presented. These will also be presented separately for endoscopic response. For each co-primary endpoint, the efficacy of each combination therapy group versus each monotherapy group will be compared, using a CMH test stratified by the randomization stratification factors.

A multiplicity-controlled testing plan will be implemented to control for the type 1 error at the 0.05 significance level (two-sided) across the multiple comparisons for the co-primary and secondary endpoints. Specifically, as part of this testing plan the high-dose combination therapy will be compared to golimumab monotherapy first, then guselkumab monotherapy on clinical remission at Week 48. If both tests are significant, the high-dose combination therapy will be compared to golimumab monotherapy, then guselkumab monotherapy on endoscopic response at Week 48. If all of these four tests are statistically significant at a 2-sided significance level of 0.05, the study will be considered positive.

The CDAI score will be calculated for a visit if at least 4 of the 8 components are available at that visit. When at least 4 of the 8 components are available, any missing components will be imputed by carrying forward the last available components. If the CDAI score cannot be calculated (ie, <4 components available) at a visit, the CDAI score will be considered missing for that visit. The total SES-CD score at a visit will be calculated based on all segments scored at the visit. If the total SES-CD score cannot be calculated (ie, no segment is scored) at a visit, the total SES-CD score will be considered missing.

After accounting for the ICE strategies, participants whose responder status is missing for a coprimary endpoint will be considered to be a non-responder for that co-primary endpoint.

To evaluate the robustness of the co-primary endpoint analyses, supplementary estimands will be evaluated and described in the SAP.

### Subgroup Analysis

To evaluate the consistency of the primary analysis, subgroup analyses based on demographics (eg, age, sex, race, body mass index, body weight, region), baseline characteristics (eg, primary non-response to an ADT, number of previous advanced therapies, baseline CRP, baseline calprotectin), disease severity (eg, baseline CDAI, baseline SES-CD, disease duration), anatomic distribution (eg, Montreal classification), and baseline medications (eg, baseline oral corticosteroid use, oral 5-ASA compounds, antibiotics for CD, and enteral nutrition) will be performed if sufficient data are available in the subgroup.

### Secondary Endpoint(s)

The following are the secondary endpoints for comparison between each combination group versus each monotherapy group:
PRO-2 remission at Week 48
Endoscopic remission at Week 48
The following are the secondary endpoints for comparison between each combination group versus placebo group: Clinical remission at Week 24
Endoscopic response at Week 24

### Estimands

### Week 48 Secondary Estimands

The attributes and strategies for the ICEs that were used for the primary estimand for the co-primary endpoint analyses will be used for the secondary endpoints of PRO-2 remission at Week 48 and endoscopic remission at Week 48 with the exception of the Variable (Endpoint), which is described as follows:
PRO-2 remission at Week 48

Variable (endpoint): Binary response variable (response/non-response) with response defined as achieved an AP mean daily score ≤1 and SF mean daily score ≤3 and no worsening of AP or SF from baseline at Week 48 without experiencing any of the ICEs in categories 1 to 5 (as outlined in the ICE attribute in the primary estimand of clinical remission at Week 48) prior to Week 48 visit.
Endoscopic remission at Week 48

Variable (endpoint): Binary variable (remission/not in remission) with remission defined as achieved endoscopic remission SES-CD score ≤4 with at least 2 points reduction from baseline and no subscore >1 in any individual component at Week 48 without experiencing any of the ICEs in categories 1-5 (as outlined in the ICE attribute in the primary estimand of clinical remission at Week 48) prior to Week 48 visit.

### Week 24 Secondary Estimands

The attributes and strategies for the ICEs (excluding ICE3 which is not applicable to Week 24 endpoints) that were used for the primary estimand for the co-primary endpoint analyses will also be used for the secondary endpoints of clinical remission at Week 24 and endoscopic response at Week 24 with the exception of the Variable (Endpoint) and Treatment, which is described as follows:
Clinical Remission at Week 24

### Treatment by Week 24:

### Experimental:

Combination high-dose group: guselkumab 320 mg and golimumab 160 mg SC co-formulation at Weeks 0, 4, and 8 followed by guselkumab 160 mg and golimumab 80 mg SC co-formulation q4w
Combination mid-dose group: guselkumab 320 mg and golimumab 160 mg SC co-formulation at Weeks 0, 4, and 8 followed by guselkumab 40 mg and golimumab 40 mg SC co-formulation q4w
Combination low-dose group: guselkumab 160 mg and golimumab 80 mg SC co-formulation at Weeks 0, 4, and 8 followed by guselkumab 20 mg and golimumab 20 mg SC co-formulation q4w

### Control:

Placebo SC at Weeks 0, 4, and 8 followed by placebo SC q4w
   Note: the guselkumab monotherapy group and the golimumab monotherapy group are not included in this estimand.

Variable (endpoint): Binary response variable (response/non-response) with response defined as achieving a CDAI score <150 at Week 24 without experiencing any of the ICEs in categories 1 to 5 (excluding ICE 3 which is not applicable to Week 24 endpoints) as outlined in Table 6, prior to the Week 24 visit.
Endoscopic response at Week 24

### Treatment by Week 24:

### Experimental:

Combination high-dose group: guselkumab 320 mg and golimumab 160 mg SC co-formulation at Weeks 0, 4, and 8 followed by guselkumab 160 mg and golimumab 80 mg SC co-formulation q4w
Combination mid-dose group: guselkumab 320 mg and golimumab 160 mg SC co-formulation at Weeks 0, 4, and 8 followed by guselkumab 40 mg and golimumab 40 mg SC co-formulation q4w
Combination low-dose group: guselkumab 160 mg and golimumab 80 mg SC co-formulation at Weeks 0, 4, and 8 followed by guselkumab 20 mg and golimumab 20 mg SC co-formulation q4w

### Control:

Placebo SC at Weeks 0, 4, and 8 followed by placebo SC q4w
   Note: the guselkumab monotherapy group and the golimumab monotherapy group are not included in this estimand.

Variable (endpoint): Binary response variable (response/non-response) with response defined as achieved a ≥50% improvement from baseline in the SES-CD score at Week 24 without experiencing any of the ICEs in categories 1 to 5 (excluding ICE 3 which is not applicable to Week 24 endpoints) as outlined in Table 6, prior to the Week 24 visit.

After accounting for the ICEs, any missing data for the secondary endpoints will be handled with non-responder imputation. To evaluate the secondary endpoints, summaries of the proportion of participants in response by treatment group, the adjusted treatment difference (with CMHstratified by randomization stratification factors) between each combination therapy group versus each monotherapy group, as well as the associated 95% confidence interval will be presented. For the testing of the secondary endpoints, the efficacy of each combination therapy group versus each monotherapy group will be compared, using a CMH test stratified by the randomization stratification factors. A multiplicity-controlled testing procedure will be implemented to control the type 1 error at the 0.05 significance level (two sided) across the primary and secondary endpoints.

### Safety Analyses

Safety data, including but not limited to, AEs, SAEs, infections, serious infections, physical examination findings, vital signs, and changes in laboratory assessments, will be summarized by System Organ Class, ADT, and treatment group for the safety analysis set, defined as all participants who received at least one dose of study intervention.

### Adverse Events

The verbatim terms used in the CRF by investigators to identify AEs will be coded using the Medical Dictionary for Regulatory Activities (MedDRA). Any AE occurring at or after the initial administration of study intervention through the day of last dose plus 12 weeks is considered to be treatment-emergent. All reported treatment-emergent AEs will be included in the analysis. For each AE, the percentage of participants who experience at least 1 occurrence of the given event will be summarized by intervention group.

The following analyses of AEs will be used to assess the safety of participants:
Frequency and type of AEs.
Frequency and type of SAEs.
Frequency and type of reasonably related AEs as assessed by the investigator.
Frequency and type of AEs leading to discontinuation of study intervention.
Frequency and type of infections, including serious infections.
Frequency and type of injection-site reactions.

Summaries, listings, datasets, or participant narratives may be provided, as appropriate, for those participants who die, who discontinue intervention due to an AE, or who experience a severe or a serious AE.

### Clinical Laboratory Tests

The following summaries of clinical laboratory tests will be used to assess participant safety:
Laboratory parameters and change from baseline in laboratory parameters (hematology and chemistry).
Summary of maximum National Cancer Institute-Common Terminology Criteria for Adverse Events (NCI-CTCAE) toxicity grade for post-baseline laboratory values (hematology and chemistry).
   Listings of participants with any abnormal post-baseline laboratory values of NCI-CTCAE grade ≥2 will also be provided.

### Suicidal Ideation and Behavior

Suicidal ideation and behavior based on the C-SSRS and AEs will be summarized descriptively.

### Other Analyses

### Pharmacokinetic Analyses

Serum guselkumab and golimumab concentrations over time will be summarized for the PK Analysis. Descriptive statistics, including arithmetic mean, SD, coefficient of variation, median, IQ range, minimum, and maximum will be calculated at each nominal sampling timepoint. All concentrations below the lowest quantifiable concentration or missing data will be labeled as such in the concentration database or data presentations. Concentrations below the lowest quantifiable concentration will be treated as zero in the summary statistics. Participants will be excluded from the PK analysis if their data do not allow for accurate assessment of the PK (eg, incomplete administration of the study intervention; missing time of study intervention administration).

Population PK modeling may be conducted when appropriate. If these population PK analyses are conducted, the results of these analyses will be presented in a separate report.

### Immunogenicity Analyses

The incidence of antibodies to guselkumab and/or to golimumab will be summarized for the Immunogenicity Analysis Set by study intervention group. A listing of participants who are positive for antibodies to guselkumab and/or to golimumab will be provided, respectively. The maximum titers of antibodies to guselkumab and/or to golimumab will be summarized for participants who are positive for antibodies to guselkumab and/or to golimumab, respectively. When there is sufficient data, persistence of antibodies to guselkumab and/or to golimumab will also be explored.

The incidence of neutralizing antibodies to guselkumab and/or to golimumab will be summarized for participants who are positive for antibodies to guselkumab and/or to golimumab and have samples evaluable for neutralizing antibodies.

### Pharmacokinetic/Pharmacodynamic Analyses

The relationship between serum concentrations of guselkumab and golimumab and efficacy measures, relevant PD endpoints, and/or safety may be explored graphically when appropriate. If any visual trend is observed, additional analysis such as exposure-response or PK/PD modeling may be conducted when appropriate. If these analyses are conducted, the results of these analyses will be presented in a separate report.

### Biomarker Analyses

Planned biomarker analyses may be deferred if emerging study data show no likelihood of providing useful scientific information. Any biomarker samples received by the contract vendor or sponsor after the cutoff date will not be analyzed, and therefore, will be excluded from the biomarker analysis.

Changes in serum protein analytes, PBMCs, histology biomarkers, stool biomarkers, biopsy, and whole blood RNA obtained over time will be summarized by treatment group. Associations between baseline levels and changes from baseline in select biomarkers and response to treatment will be explored. Biomarker analyses will be summarized in a separate technical report.

The biomarker analyses will characterize the effects of guselkumab and golimumab monotherapy and combination therapy to identify biomarkers relevant to treatment, and to determine if these biomarkers can predict response to either guselkumab or golimumab monotherapy or combination therapy. Results of whole blood, exploratory serum, PBMCs, ileocolonic biopsy, and stool biomarker analyses will be reported in separate technical reports.

### Pharmacogenomic Analyses

Any additional genetic analyses will be considered exploratory.

### Medical Resource Utilization and Health Economic Analyses

Medical resource utilization, including but not limited to CD-related emergency department visits, hospitalizations, and surgeries, will be collected in this study. The data will be descriptively summarized by intervention group.

The Work Productivity and Activity Impairment-Crohn's Disease (WPAI-CD) will also be utilized to compare work productivity and activity impairment and will be based on the study intervention group.

### Interim Analyses

One interim analyses (IA) is planned in the study. The IA will be a futility analysis to be conducted after the first 250 randomized and treated participants (approximately 35%) have reached Week 12 (or have terminated study participation prior to the Week 12 visit) and at least the first 110 randomized and treated participants have reached Week 24 (or have terminated study participation before the Week 24 visit).

The planned IA will not cause alpha inflation because there is no plan for an early efficacy stop or adaptation to the remainder of the study, other than terminating the study based on futility. Therefore, a 2-sided alpha=0.05 can be used in the final analysis. Posterior probabilities will be used in the IA to assist decision-making if deemed reasonable. Details regarding the non-binding futility rule will be provided in the Interim Analysis Plan (IAP).

The IA will be handled in a manner such that the integrity of the study will be preserved. An external SSG will perform the IA. The DMC will review the IA results and form a recommendation on whether to stop the study for futility. The Sponsor Committee will then review the DMC's recommendation and make a final decision.

The sponsor will not have access to treatment assignment information at the time of the IA. The sponsor will also not have access to the futility results unless the DMC recommends stopping the study for futility or further evaluation is needed, in which case the Sponsor Committee may request unblinded information for making the final decision. If the Sponsor Committee does receive the IA results, the confidentiality of these results will be maintained for the remainder of the study.

### The Invention can be Described with Reference to the Following Numbered Embodiments

1. A combination of an IL-23 inhibitor and a TNF-α inhibitor for use in the treatment of Crohn's disease (CD) in a patient, wherein the use results in a clinical response in the patient.
2. A combination of an IL-23 inhibitor and a TNF-α inhibitor for use according to embodiment 1, wherein the IL-23 inhibitor comprises an anti-IL-23p19 antibody or an antigen-binding fragment thereof and the TNF-α inhibitor comprises an anti-TNF-α antibody or an antigen-binding fragment thereof.
3. A combination of an IL-23 inhibitor and a TNF-α inhibitor for use according to embodiment 1, wherein the IL-23 inhibitor is selected from the group consisting of guselkumab, risankizumab, tildrakizumab and mirikizumab, and the TNF-α inhibitor is selected from the group consisting of golimumab, adalimumab, infliximab, certolizumab pegol and etanercept.
4. A combination of an IL-23 inhibitor and a TNF-α inhibitor for use according to embodiment 2, wherein the anti-IL-23p19 antibody comprises: a) heavy chain complementarity determining region (CDR) amino acid sequences of SEQ ID NOs: 1-3 and light chain CDR amino acid sequences of SEQ ID NOs: 4-6; b) a heavy chain variable region amino acid sequence of SEQ ID NO: 7 and a light chain variable region amino acid sequence of SEQ ID NO: 8; or c) a heavy chain amino acid sequence of SEQ ID NO: 9 and a light chain amino acid sequence of SEQ ID NO: 10.
5. A combination of an IL-23 inhibitor and a TNF-α inhibitor for use according to embodiment 2, wherein the anti-TNF-α antibody comprises: a) heavy chain CDR amino acid sequences of SEQ ID NOs: 11-13 and light chain CDR amino acid sequences of SEQ ID NOs: 14-16; b) a heavy chain variable region amino acid sequence of SEQ ID NO: 17 and a light chain variable region amino acid sequence of SEQ ID NO: 18; or c) a heavy chain amino acid sequence of SEQ ID NO: 19 and a light chain amino acid sequence of SEQ ID NO: 20.
6. A combination of an IL-23 inhibitor and a TNF-α inhibitor for use according to embodiment 2, wherein the anti-IL-23p19 antibody comprises: a) heavy chain CDR amino acid sequences of SEQ ID NOs: 1-3 and light chain CDR amino acid sequences of SEQ ID NOs: 4-6; b) a heavy chain variable region amino acid sequence of SEQ ID NO: 7 and a light chain variable region amino acid sequence of SEQ ID NO: 8; or c) a heavy chain amino acid sequence of SEQ ID NO: 9 and a light chain amino acid sequence of SEQ ID NO: 10, and the anti-TNF-α antibody comprises: a) heavy chain CDR amino acid sequences of SEQ ID NOs: 11-13 and light chain CDR amino acid sequences of SEQ ID NOs: 14-16; b) a heavy chain variable region amino acid sequence of SEQ ID NO: 17 and a light chain variable region amino acid sequence of SEQ ID NO: 18; or c) a heavy chain amino acid sequence of SEQ ID NO: 19 and a light chain amino acid sequence of SEQ ID NO: 20.
7. A combination of an IL-23 inhibitor and a TNF-α inhibitor for use according to any one of the preceding embodiments, wherein the combination comprises the IL-23 inhibitor and the TNF-α inhibitor at a weight ratio of from about 2:1 to 1:2.
8. A combination of an IL-23 inhibitor and a TNF-α inhibitor for use according to any one of the preceding embodiments, wherein the combination comprises the IL-23 inhibitor and the TNF-α inhibitor formulated in separate syringes and administered subcutaneously.
9. A combination of an IL-23 inhibitor and a TNF-α inhibitor for use according to any one of the preceding embodiments, wherein, i) the combination comprises the IL-23 inhibitor and the TNF-α inhibitor co-formulated in a single syringe and administered subcutaneously in a single administration or ii) the combination comprises the IL-23 inhibitor and the TNF-α inhibitor separately formulated in separate syringes and mixed and administered subcutaneously in a single administration.
10. A combination of an IL-23 inhibitor and a TNF-α inhibitor for use according to any one of the preceding embodiments, wherein the combination comprises about 20-1000 mg of the IL-23 inhibitor and about 20-1000 mg of the TNF-α inhibitor and is administered subcutaneously every 1, 2, 3, 4, 5, 6, 7, or 8 weeks.
11. A combination of an IL-23 inhibitor and a TNF-α inhibitor for use according to any one of the preceding embodiments, wherein the combination comprises about 320 mg of the IL-23 inhibitor and about 160 mg of the TNF-α inhibitor and is administered subcutaneously at weeks 0, 4, and 8.
12. A combination of an IL-23 inhibitor and a TNF-α inhibitor for use according to embodiment 11, further comprising administering subcutaneously about 160 mg of the IL-23 inhibitor and about 80 mg of the TNF-α inhibitor every 4 weeks after week 8.
13. A combination of an IL-23 inhibitor and a TNF-α inhibitor for use according to embodiment 11, further comprising administering subcutaneously about 40 mg of the IL-23 inhibitor and about 40 mg of the TNF-α inhibitor every 4 weeks after week 8.
14. A combination of an IL-23 inhibitor and a TNF-α inhibitor for use according to any one of embodiments 1-10, wherein the combination comprises about 160 mg of the IL-23 inhibitor and about 80 mg of the TNF-α inhibitor and is administered subcutaneously at weeks 0, 4, and 8.
15. A combination of an IL-23 inhibitor and a TNF-α inhibitor for use according to embodiment 14, further comprising administering subcutaneously about 20 mg of the IL-23 inhibitor and about 20 mg of the TNF-α inhibitor every 4 weeks after week 8.
16. A combination of an IL-23 inhibitor and a TNF-α inhibitor for use according to any one of embodiments 1-10, comprising administering subcutaneously (i) about 160 mg of the IL-23 inhibitor and about 80 mg of the TNF-α inhibitor every 4 weeks, (ii) about 40 mg of the IL-23 inhibitor and about 40 mg of the TNF-α inhibitor every 4 weeks, or (iii) about 20 mg of the IL-23 inhibitor and about 20 mg of the TNF-α inhibitor every 4 weeks.
17. A combination of an IL-23 inhibitor and a TNF-α inhibitor for use according to any one of the preceding embodiments, wherein the patient suffers moderately or severely active CD.
18. A combination of an IL-23 inhibitor and a TNF-α inhibitor for use according to embodiment 17, wherein the patient was previously treated with an ADT, for example, a TNF-α inhibitor alone and wherein the CD did not undergo remission after the previous treatment.
19. A combination of an IL-23 inhibitor and a TNF-α inhibitor for use according to embodiment claim 17, wherein the patient was previously treated with an ADT, for example an IL-23 inhibitor alone and wherein the CD did not undergo remission after the previous treatment.
20. A combination of an IL-23 inhibitor and a TNF-α inhibitor for use according to embodiment 17, wherein the patient was previously treated with an ADT, for example a TNF-α inhibitor or an IL-23 inhibitor alone, and wherein the patient had an inadequate initial clinical response, loss of clinical response, or intolerance to ≥1 previous ADT.
21. A combination of an IL-23 inhibitor and a TNF-α inhibitor for use according to any one of the preceding embodiments, wherein the clinical response is based on a clinical endpoint selected from the group consisting of: (i) achievement of Crohn's Disease Activity Index (CDAI) score <150, (ii) achievement of a ≥50% improvement from baseline in Simple Endoscopic Score for Crohn's Disease (SES-CD) score, (iii) achievement of an abdominal pain (AP) mean daily score ≤1 and stool frequency (SF) mean daily score ≤3, and no worsening of AP or SF from baseline, and (iv) achievement of SES-CD≤2 or SES-CD score ≤4 with at least 2 points reduction from baseline and no subscore >1 in any individual component.
22. A combination of an IL-23 inhibitor and a TNF-α inhibitor for use according to any one of embodiments 1-21, wherein the clinical response is measured about 24 weeks, 48 weeks, or 240 weeks after initial treatment.
23. A combination of an IL-23 inhibitor and a TNF-α inhibitor for use according to any one of the preceding embodiments, wherein the method is clinically safe in treating the patient, or wherein the method results in a reduced adverse effect compared to a treatment with a TNF-α inhibitor alone or an IL-23 inhibitor alone.
24. A kit comprising (1) an IL-23 inhibitor and a TNF-α inhibitor, and (2) instructions for treating CD in a patient, wherein the instructions comprise subcutaneously administering to the patient (i) about 320 mg of the IL-23 inhibitor and about 160 mg of the TNF-α inhibitor at weeks 0, 4, and 8; (ii) about 160 mg of the IL-23 inhibitor and about 80 mg of the TNF-α inhibitor at weeks 0, 4, and 8; (iii) about 160 mg of the IL-23 inhibitor and about 80 mg of the TNF-α inhibitor every 4 weeks; (iv) about 40 mg of the IL-23 inhibitor and about 40 mg of the TNF-α inhibitor every 4 weeks; or (v) about 20 mg of the IL-23 inhibitor and about 20 mg of the TNF-α inhibitor every 4 weeks.
25. A combination of an anti-IL-23p19 antibody and an anti-TNF-α antibody for use in the treatment of CD in a patent, wherein,
   a) the anti-IL-23p19 antibody comprises (i) the heavy chain CDR amino acid sequences of SEQ ID NOs: 1-3 and the light chain CDR amino acid sequences of SEQ ID NOs: 4-6, (ii) the heavy chain variable region amino acid sequence of SEQ ID NO: 7 and the light chain variable region amino acid sequence of SEQ ID NO: 8, or (iii) the heavy chain amino acid sequence of SEQ ID NO: 9 and the light chain amino acid sequence of SEQ ID NO: 10;
   b) the anti-TNF-α antibody comprises (i) the heavy chain CDR amino acid sequences of SEQ ID NOs: 11-13 and the light chain CDR amino acid sequences of SEQ ID NOs: 14-16, (ii) the heavy chain variable region amino acid sequence of SEQ ID NO: 17 and the light chain variable region amino acid sequence of SEQ ID NO: 18, or (iii) the heavy chain amino acid sequence of SEQ ID NO: 19 and the light chain amino acid sequence of SEQ ID NO:20; and
   c) the method results in a clinical response in the patient and the clinical response is based on a clinical endpoint selected from the group consisting of: (i) achievement of Crohn's Disease Activity Index (CDAI) score <150, (ii) achievement of a ≥50% improvement from baseline in Simple Endoscopic Score for Crohn's Disease (SES-CD) score, (iii) achievement of an abdominal pain (AP) mean daily score ≤1 and stool frequency (SF) mean daily score ≤3, and no worsening of the AP or SF from baseline, and (iv) achievement of SES-CD≤2 or SES-CD score ≤4 with at least 2 points reduction from baseline and no subscore >1 in any individual component.
26. A combination of an anti-IL-23p19 antibody and an anti-TNF-α antibody for use according to embodiment 25, wherein the combination comprises the anti-IL-23p19 antibody and the anti-TNF-α antibody at a weight ratio of from about 2:1 to 1:2.
27. A combination of an anti-IL-23p19 antibody and an anti-TNF-α antibody for use according to embodiment 25 or 26, wherein the combination comprises the anti-IL-23p19 antibody and the anti-TNF-α antibody formulated in separate syringes and administered subcutaneously.
28. A combination of an anti-IL-23p19 antibody and an anti-TNF-α antibody for use according to embodiment 25 or 26, wherein, i) the combination comprises the anti-IL-23p19 antibody and the anti-TNF-α antibody co-formulated in a single syringe and administered subcutaneously in a single administration or ii) the combination comprises the anti-IL-23p19 antibody and the anti-TNF-α antibody separately formulated in separate syringes and mixed and administered subcutaneously in a single administration.
29. A combination of an anti-IL-23p19 antibody and an anti-TNF-α antibody for use according to any one of embodiments 25-28, wherein the combination comprises about 20-1000 mg of the anti-IL-23 antibody and about 20-1000 mg of the anti-TNF-α antibody and is administered subcutaneously every 1, 2, 3, 4, 5, 6, 7, or 8 weeks.
30. A combination of an anti-IL-23p19 antibody and an anti-TNF-α antibody for use according to any one of embodiments 25-29, wherein the combination comprises about 320 mg of the anti-IL-23 antibody and about 160 mg of the anti-TNF-α antibody and is administered subcutaneously at weeks 0, 4, and 8.
31. A combination of an anti-IL-23p19 antibody and an anti-TNF-α antibody for use according to embodiment 30, further comprising administering subcutaneously about 160 mg of the anti-IL-23p19 antibody and about 80 mg of the anti-TNF-α antibody every 4 weeks after week 8.
32. A combination of an anti-IL-23p19 antibody and an anti-TNF-α antibody for use according to embodiment 30, further comprising administering subcutaneously about 40 mg of the anti-IL-23p19 antibody and about 40 mg of the anti-TNF-α antibody every 4 weeks after week 8.
33. A combination of an anti-IL-23p19 antibody and an anti-TNF-α antibody for use according to any one of embodiments 25-29, wherein the combination comprises about 160 mg of the anti-IL-23p19 antibody and about 80 mg of the anti-TNF-α antibody and is administered subcutaneously at weeks 0, 4, and 8.
34. A combination of an anti-IL-23p19 antibody and an anti-TNF-α antibody for use according to embodiment claim 33, further comprising administering subcutaneously about 20 mg of the anti-IL-23 antibody and about 20 mg of the anti-TNF-α antibody every 4 weeks after week 8.
35. A combination of an anti-IL-23p19 antibody and an anti-TNF-α antibody for use according to any one of embodiments 25-29, comprising administering subcutaneously (i) about 160 mg of the anti-IL-23p19 antibody and about 80 mg of the anti-TNF-α antibody every 4 weeks, (ii) about 40 mg of the anti-IL-23p19 antibody and about 40 mg of the anti-TNF-α antibody every 4 weeks, or (iii) about 20 mg of the anti-IL-23p19 antibody and about 20 mg of the anti-TNF-α antibody every 4 weeks.
36. A combination of an anti-IL-23p19 antibody and an anti-TNF-α antibody for use according to any one of embodiments 25-35, wherein the patient suffers moderately or severely active CD.
37. A combination of an anti-IL-23p19 antibody and an anti-TNF-α antibody for use according to embodiment 36, wherein the patient was previously treated with an ADT, for example, a TNF-α inhibitor alone and wherein the CD did not undergo remission after the previous treatment.
38. A combination of an anti-IL-23p19 antibody and an anti-TNF-α antibody for use according to embodiment 36, wherein the patient was previously treated with an ADT, for example, an IL-23 inhibitor alone and wherein the CD did not undergo remission after the previous treatment.
39. A combination of an anti-IL-23p19 antibody and an anti-TNF-α antibody for use according to embodiment 36, wherein the patient was previously treated with an ADT, for example, a TNF-α inhibitor or an IL-23 inhibitor alone, and wherein the patient had an inadequate initial clinical response, loss of clinical response, or intolerance to ≥1 previous ADT.
40. A combination of an anti-IL-23p19 antibody and an anti-TNF-α antibody for use according to any one of embodiments 25-39, wherein the method is clinically safe in treating the patient, or wherein the method results in a reduced adverse effect compared to a treatment with a TNF-α inhibitor alone or an IL-23 inhibitor alone.

1. A method of treating Crohn's disease (CD) in a patient, the method comprising administering a combination of an IL-23 inhibitor and a TNF-α inhibitor, wherein the method results in a clinical response in the patient.

2. The method of claim 1, wherein the IL-23 inhibitor comprises an anti-IL-23p19 antibody or an antigen-binding fragment thereof and the TNF-α inhibitor comprises an anti-TNF-α antibody or an antigen-binding fragment thereof.

3. The method of claim 1, wherein the IL-23 inhibitor is selected from the group consisting of guselkumab, risankizumab, tildrakizumab and mirikizumab, and the TNF-α inhibitor is selected from the group consisting of golimumab, adalimumab, infliximab, certolizumab pegol and etanercept.

4. The method of claim 2, wherein the anti-IL-23p19 antibody comprises: a) heavy chain complementarity determining region (CDR) amino acid sequences of SEQ ID NOs: 1-3 and light chain CDR amino acid sequences of SEQ ID NOs: 4-6; b) a heavy chain variable region amino acid sequence of SEQ ID NO: 7 and a light chain variable region amino acid sequence of SEQ ID NO: 8; or c) a heavy chain amino acid sequence of SEQ ID NO: 9 and a light chain amino acid sequence of SEQ ID NO: 10.

5. The method of claim 2, wherein the anti-TNF-α antibody comprises: a) heavy chain CDR amino acid sequences of SEQ ID NOs: 11-13 and light chain CDR amino acid sequences of SEQ ID NOs: 14-16; b) a heavy chain variable region amino acid sequence of SEQ ID NO: 17 and a light chain variable region amino acid sequence of SEQ ID NO: 18; or c) a heavy chain amino acid sequence of SEQ ID NO: 19 and a light chain amino acid sequence of SEQ ID NO: 20.

6. The method of claim 2, wherein the anti-IL-23p19 antibody comprises: a) heavy chain CDR amino acid sequences of SEQ ID NOs: 1-3 and light chain CDR amino acid sequences of SEQ ID NOs: 4-6; b) a heavy chain variable region amino acid sequence of SEQ ID NO: 7 and a light chain variable region amino acid sequence of SEQ ID NO: 8; or c) a heavy chain amino acid sequence of SEQ ID NO: 9 and a light chain amino acid sequence of SEQ ID NO: 10, and the anti-TNF-α antibody comprises: a) heavy chain CDR amino acid sequences of SEQ ID NOs: 11-13 and light chain CDR amino acid sequences of SEQ ID NOs: 14-16; b) a heavy chain variable region amino acid sequence of SEQ ID NO: 17 and a light chain variable region amino acid sequence of SEQ ID NO: 18; or c) a heavy chain amino acid sequence of SEQ ID NO: 19 and a light chain amino acid sequence of SEQ ID NO: 20.

7. The method of any one of claims 1-6, wherein the combination comprises the IL-23 inhibitor and the TNF-α inhibitor at a weight ratio of from about 2:1 to 1:2.

8. The method of any one of claims 1-7, wherein the combination comprises the IL-23 inhibitor and the TNF-α inhibitor formulated in separate syringes and administered subcutaneously.

9. The method of any one of claims 1-7, wherein, i) the combination comprises the IL-23 inhibitor and the TNF-α inhibitor co-formulated in a single syringe and administered subcutaneously in a single administration or ii) the combination comprises the IL-23 inhibitor and the TNF-α inhibitor separately formulated in separate syringes and mixed and administered subcutaneously in a single administration.

10. The method of any one of claims 1-9, wherein the combination comprises about 20-1000 mg of the IL-23 inhibitor and about 20-1000 mg of the TNF-α inhibitor and is administered subcutaneously every 1, 2, 3, 4, 5, 6, 7, or 8 weeks.

11. The method of any one of claims 1-10, wherein the combination comprises about 320 mg of the IL-23 inhibitor and about 160 mg of the TNF-α inhibitor and is administered subcutaneously at weeks 0, 4, and 8.

12. The method of claim 11, further comprising administering subcutaneously about 160 mg of the IL-23 inhibitor and about 80 mg of the TNF-α inhibitor every 4 weeks after week 8.

13. The method of claim 11, further comprising administering subcutaneously about 40 mg of the IL-23 inhibitor and about 40 mg of the TNF-α inhibitor every 4 weeks after week 8.

14. The method of any one of claims 1-10, wherein the combination comprises about 160 mg of the IL-23 inhibitor and about 80 mg of the TNF-α inhibitor and is administered subcutaneously at weeks 0, 4, and 8.

15. The method of claim 14, further comprising administering subcutaneously about 20 mg of the IL-23 inhibitor and about 20 mg of the TNF-α inhibitor every 4 weeks after week 8.

16. The method of any one of claims 1-10, comprising administering subcutaneously (i) about 160 mg of the IL-23 inhibitor and about 80 mg of the TNF-α inhibitor every 4 weeks, (ii) about 40 mg of the IL-23 inhibitor and about 40 mg of the TNF-α inhibitor every 4 weeks, or (iii) about 20 mg of the IL-23 inhibitor and about 20 mg of the TNF-α inhibitor every 4 weeks.

17. The method of any one of claims 1-16, wherein the patient suffers moderately or severely active CD.

18. The method of claim 17, wherein the patient was previously treated with an ADT, for example, a TNF-α inhibitor alone and wherein the CD did not undergo remission after the previous treatment.

19. The method of claim 17, wherein the patient was previously treated with an ADT, for example, an IL-23 inhibitor alone and wherein the CD did not undergo remission after the previous treatment.

20. The method of claim 17, wherein the patient was previously treated with an ADT, for example, a TNF-α inhibitor or an IL-23 inhibitor alone, and wherein the patient had an inadequate initial clinical response, loss of clinical response, or intolerance to ≥1 previous ADT.

21. The method of any one of claims 1-20, wherein the clinical response is based on a clinical endpoint selected from the group consisting of: (i) achievement of Crohn's Disease Activity Index (CDAI) score <150, (ii) achievement of a ≥50% improvement from baseline in Simple Endoscopic Score for Crohn's Disease (SES-CD) score, (iii) achievement of an abdominal pain (AP) mean daily score ≤1 and stool frequency (SF) mean daily score ≤3, and no worsening of the AP or SF from baseline, and (iv) achievement of SES-CD≤2 or SES-CD score ≤4 with at least 2 points reduction from baseline and no subscore >1 in any individual component.

22. The method of any one of claims 1-21, wherein the clinical response is measured about 24 weeks, 48 weeks, or 240 weeks after initial treatment.

23. The method of any one of claims 1-22, wherein the method is clinically safe in treating the patient, or wherein the method results in a reduced adverse effect compared to a treatment with a TNF-α inhibitor alone or an IL-23 inhibitor alone.

24. A kit comprising (1) an IL-23 inhibitor and a TNF-α inhibitor, and (2) instructions for treating CD in a patient, wherein the instructions comprise subcutaneously administering to the patient (i) about 320 mg of the IL-23 inhibitor and about 160 mg of the TNF-α inhibitor at weeks 0, 4, and 8; (ii) about 160 mg of the IL-23 inhibitor and about 80 mg of the TNF-α inhibitor at weeks 0, 4, and 8; (iii) about 160 mg of the IL-23 inhibitor and about 80 mg of the TNF-α inhibitor every 4 weeks; (iv) about 40 mg of the IL-23 inhibitor and about 40 mg of the TNF-α inhibitor every 4 weeks; or (v) about 20 mg of the IL-23 inhibitor and about 20 mg of the TNF-α inhibitor every 4 weeks.

25. A method of treating CD in a patent, the method comprising administering a combination of an anti-IL-23p19 antibody and an anti-TNF-α antibody, wherein,
a) the anti-IL-23p19 antibody comprises (i) the heavy chain CDR amino acid sequences of SEQ ID NOs: 1-3 and the light chain CDR amino acid sequences of SEQ ID NOs: 4-6, (ii) the heavy chain variable region amino acid sequence of SEQ ID NO: 7 and the light chain variable region amino acid sequence of SEQ ID NO: 8, or (iii) the heavy chain amino acid sequence of SEQ ID NO: 9 and the light chain amino acid sequence of SEQ ID NO: 10;
b) the anti-TNF-α antibody comprises (i) the heavy chain CDR amino acid sequences of SEQ ID NOs: 11-13 and the light chain CDR amino acid sequences of SEQ ID NOs: 14-16, (ii) the heavy chain variable region amino acid sequence of SEQ ID NO: 17 and the light chain variable region amino acid sequence of SEQ ID NO: 18, or (iii) the heavy chain amino acid sequence of SEQ ID NO: 19 and the light chain amino acid sequence of SEQ ID NO:20; and
c) the method results in a clinical response in the patient and the clinical response is based on a clinical endpoint selected from the group consisting of: (i) achievement of Crohn's Disease Activity Index (CDAI) score <150, (ii) achievement of a ≥50% improvement from baseline in Simple Endoscopic Score for Crohn's Disease (SES-CD) score, (iii) achievement of an abdominal pain (AP) mean daily score ≤1 and stool frequency (SF) mean daily score ≤3, and no worsening of the AP or SF from baseline, and (iv) achievement of SES-CD≤2 or SES-CD score ≤4 with at least 2 points reduction from baseline and no subscore >1 in any individual component.

26. The method of claim 25, wherein the combination comprises the anti-IL-23p19 antibody and the anti-TNF-α antibody at a weight ratio of from about 2:1 to 1:2.

27. The method of claim 25 or 26, wherein the combination comprises the anti-IL-23p19 antibody and the anti-TNF-α antibody formulated in separate syringes and administered subcutaneously.

28. The method of claim 25 or 26, wherein, i) the combination comprises the anti-IL-23p19 antibody and the anti-TNF-α antibody co-formulated in a single syringe and administered subcutaneously in a single administration or ii) the combination comprises the anti-IL-23p19 antibody and the anti-TNF-α antibody separately formulated in separate syringes and mixed and administered subcutaneously in a single administration.

29. The method of any one of claims 25-28, wherein the combination comprises about 20-1000 mg of the anti-IL-23 antibody and about 20-1000 mg of the anti-TNF-α antibody and is administered subcutaneously every 1, 2, 3, 4, 5, 6, 7, or 8 weeks.

30. The method of any one of claims 25-29, wherein the combination comprises about 320 mg of the anti-IL-23 antibody and about 160 mg of the anti-TNF-α antibody and is administered subcutaneously at weeks 0, 4, and 8.

31. The method of claim 30, further comprising administering subcutaneously about 160 mg of the anti-IL-23p19 antibody and about 80 mg of the anti-TNF-α antibody every 4 weeks after week 8.

32. The method of claim 30, further comprising administering subcutaneously about 40 mg of the anti-IL-23p19 antibody and about 40 mg of the anti-TNF-α antibody every 4 weeks after week 8.

33. The method of any one of claims 25-29, wherein the combination comprises about 160 mg of the anti-IL-23p19 antibody and about 80 mg of the anti-TNF-α antibody and is administered subcutaneously at weeks 0, 4, and 8.

34. The method of claim 33, further comprising administering subcutaneously about 20 mg of the anti-IL-23 antibody and about 20 mg of the anti-TNF-α antibody every 4 weeks after week 8.

35. The method of any one of claims 25-29, comprising administering subcutaneously (i) about 160 mg of the anti-IL-23p19 antibody and about 80 mg of the anti-TNF-α antibody every 4 weeks, (ii) about 40 mg of the anti-IL-23p19 antibody and about 40 mg of the anti-TNF-α antibody every 4 weeks, or (iii) about 20 mg of the anti-IL-23p19 antibody and about 20 mg of the anti-TNF-α antibody every 4 weeks.

36. The method of any one of claims 25-35, wherein the patient suffers moderately or severely active CD.

37. The method of claim 36, wherein the patient was previously treated with an ADT, for example, a TNF-α inhibitor alone and wherein the CD did not undergo remission after the previous treatment.

38. The method of claim 36, wherein the patient was previously treated with an ADT, for example, an IL-23 inhibitor alone and wherein the CD did not undergo remission after the previous treatment.

39. The method of claim 36, wherein the patient was previously treated with an ADT, for example, a TNF-α inhibitor or an IL-23 inhibitor alone, and wherein the patient had an inadequate initial clinical response, loss of clinical response, or intolerance to ≥1 previous ADT.

40. The method of any one of claims 25-39, wherein the method is clinically safe in treating the patient, or wherein the method results in a reduced adverse effect compared to a treatment with a TNF-α inhibitor alone or an IL-23 inhibitor alone.
